(19) United States
(12) Patent Application Publication
(10) Pub. No.: US 2026/0083348 A1
(43) Pub. Date: Mar. 26, 2026

(54) SENSORY ARTIFICIAL CILIA FOR IN SITU MONITORING OF AIRWAY PHYSIOLOGICAL PROPERTIES

(71) Applicant: VANDERBILT UNIVERSITY, Nashville, TN (US)

(72) Inventors: Xiaoguang Dong, Nashville, TN (US); Yusheng Wang, Nashville, TN (US)

(21) Appl. No.: 19/336,708

(22) Filed: Sep. 23, 2025

Related U.S. Application Data
(60) Provisional application No. 63/699,030, filed on Sep. 25, 2024.

(51) Int. Cl. A61B 5/08; A61B 5/00; A61B 5/145; A61F 2/04

(52) U.S. Cl. CPC A61B 5/08; A61B 5/14507; A61B 5/746; A61F 2/04; A61B 2560/0214; A61B 2560/0223; A61B 2562/0261; A61F 2002/046

(57) ABSTRACT

The present disclosure provides for sensory artificial cilia for in situ monitoring of airway physiological properties, methods of monitoring an airway of a subject, and systems including an implant and a magnetic actuator configured to produce an external magnetic field which actuates the implant to measure a viscosity and a thickness of a fluid within an airway of a subject. In various aspects, a viscosity sensor of the implant can include a strain-gauge configured to measure a deformation of the viscosity sensor based at least in part on an electrical resistance of the strain-gauge, where the viscosity of the fluid is calculated based at least in part on the deformation. In various aspects, a layer thickness sensor of the implant can include a capacitor configured to measure a capacitance of the capacitor, where the thickness of the fluid is calculated based at least in part on the capacitance.

### CROSS-REFERENCE TO RELATED APPLICATIONS

This application claims the benefit of U.S. Provisional Application entitled “SENSORY ARTIFICIAL CILIA FOR IN SITU MONITORING OF AIRWAY PHYSIOLOGICAL PROPERTIES” and having Ser. No. 63/699,030, filed Sep. 25, 2024, which is herein incorporated by reference in its entirety.

### BACKGROUND

Continuously monitoring human airway conditions is important for timely interventions, especially when airway stents are implanted to alleviate central airway obstruction in lung cancer and other diseases. Mucus conditions can be important biomarkers for indicating inflammation and stent patency but can be challenging to monitor. Airway stents can be used to address diseases like central airway obstruction, as seen in conditions such as lung cancer, wherein the airway narrows from extrinsic compression. However, a drawback of current airway stents can be interference with airway cilia, microscopic structures responsible for mucus transport, leading to mucus build-up. The accumulation of thick mucus in the airways poses challenges for patients by obstructing breathing and inducing inflammation, resulting in unpredictable episodes of airway distress and urgent bronchoscopy in cases of excessive mucus accumulation. Current monitoring methods often cannot provide continuous monitoring and rely on scheduled medical imaging checks. For example, computational tomography uses radiation, is expensive, and is performed in hospitals. In addition, bronchoscopy can be used for follow-up stent patency checks but is performed under anesthesia.

### SUMMARY

In accordance with the purpose(s) of this disclosure, as embodied and broadly described herein, the disclosure, in various aspects, relates to sensory artificial cilia for in situ monitoring of airway physiological properties and methods of use thereof.

Aspects of the present disclosure provide for a sensory artificial cilia for in situ monitoring of airway physiological properties. Embodiments of the present disclosure include: a device, including a tubular member having a radially external side and a radially internal side. The device can include a viscosity sensor configured to measure a viscosity of a fluid within an airway of a subject in response to a bending motion of the viscosity sensor and a layer thickness sensor configured to measure a thickness of the fluid within the airway of the subject in response to a tilting motion of the layer thickness sensor. The layer thickness sensor can be configured to engage in the tilting motion, the viscosity sensor can be configured to engage in the bending motion, or both in response to an application of an external magnetic field. In various aspects, the device can include one or more of a temperature sensor or a magnetic sensor disposed on the radially internal side of the tubular member. In some aspects, the device can include a wireless communication module disposed on the radially internal side of the tubular member and configured to wirelessly communicate with one or more computing devices. In some aspects, the device can include an airway stent coupled to one or more ends of the tubular member.

In various aspects, the external magnetic field can have a frequency of approximately 0.01 Hz to approximately 10 Hz. In some aspects, the external magnetic field can have a frequency of approximately 0.5 Hz to approximately 8 Hz. In a preferred aspect, the external magnetic field can have a frequency of approximately 0.1 Hz to approximately 5 Hz. In various aspects, the external magnetic field can have a magnitude of approximately 1 mT to approximately 100 mT. In some aspects, the external magnetic field can have a magnitude of approximately 2 mT to approximately 80 mT. In a preferred aspect, the external magnetic field can have a magnitude of approximately 5 mT to approximately 70 mT.

In various aspects, the viscosity sensor can include a strain-gauge configured to measure a deformation of the viscosity sensor based at least in part on an electrical resistance of the strain-gauge, where the viscosity of the fluid is calculated based at least in part on the deformation. In various aspects, the layer thickness sensor can include a capacitor configured to measure a capacitance of the capacitor, where the thickness of the fluid is calculated based at least in part on the capacitance. In some aspects, the device can include at least one rechargeable battery disposed on the radially internal side of the tubular member, where the application of the external magnetic field charges the at least one rechargeable battery.

The present disclosure also provides a method of monitoring an airway of a subject, including the steps of applying an external magnetic field to an artificial cilium implanted within the airway of the subject, where the artificial cilium includes a layer thickness sensor, measuring a capacitance using the layer thickness sensor, and calculating a thickness of a fluid in the airway of the subject based at least in part on the capacitance.

In various aspects, the external magnetic field can have a frequency of approximately 0.01 Hz to approximately 10 Hz. In some aspects, the external magnetic field can have a frequency of approximately 0.5 Hz to approximately 8 Hz. In a preferred aspect, the external magnetic field can have a frequency of approximately 0.1 Hz to approximately 5 Hz. In various aspects, the external magnetic field can have a magnitude of approximately 1 mT to approximately 100 mT. In some aspects, the external magnetic field can have a magnitude of approximately 2 mT to approximately 80 mT. In a preferred aspect, the external magnetic field can have a magnitude of approximately 5 mT to approximately 70 mT.

In some aspects, the artificial cilium can include a viscosity sensor, and the method can further include determining the thickness of the fluid is greater than a threshold, where the threshold is based at least in part on the viscosity sensor, applying a second external magnetic field, measuring a deformation of the viscosity sensor, and calculating a viscosity of the fluid in the airway of the subject based at least in part on the deformation.

In various aspects, the second external magnetic field can have a frequency of approximately 0.01 Hz to approximately 10 Hz. In some aspects, the second external magnetic field can have a frequency of approximately 0.5 Hz to approximately 8 Hz. In a preferred aspect, the second external magnetic field can have a frequency of approximately 0.1 Hz to approximately 5 Hz. In various aspects, the second external magnetic field can have a magnitude of approximately 1 mT to approximately 100 mT. In some aspects, the second external magnetic field can have a magnitude of approximately 2 mT to approximately 80 mT. In a preferred aspect, the second external magnetic field can have a magnitude of approximately 5 mT to approximately 70 mT.

In various aspects, measuring the capacitance using the layer thickness sensor can include measuring a maximum capacitance without the application of the external magnetic field, applying the external magnetic field to tilt the layer thickness sensor to a tilting angle, where the tilting angle is based at least in part on an angle of the external magnetic field, and gradually increasing the tilting angle until the capacitance reaches the maximum capacitance. In some aspects, the method can further include diagnosing a condition of the airway of the subject based at least in part on the thickness of the fluid, the viscosity of the fluid, or both. In some aspects, the method can further include alerting a user when the thickness of the fluid is equal to or greater than a threshold.

Additionally, the present disclosure provides a system, including an implant, a magnetic actuator, and a device. In various aspects, the device can be any device capable of activating the magnetic actuation device to tilt the layer thickness sensor based at least in part on a first external magnetic field, calculating, using the layer thickness sensor, the thickness of the fluid within the airway of the subject based at least in part on the application of the first external magnetic field, activating the magnetic actuation device to bend the viscosity sensor based at least in part on a second external magnetic field, and calculating, using the viscosity sensor, the viscosity of the fluid within the airway of the subject based at least in part on the application of the second external magnetic field. In an aspect, the device can be a computing device in wireless communication with the implant and the magnetic actuation device, including a processor and a memory, and machine-readable instructions stored in the memory. The implant can include a tubular member having a radially external side and a radially internal side, a viscosity sensor disposed on the radially internal side of the tubular member and configured to measure a viscosity of a fluid in an airway of a subject in response to a bending motion of the viscosity sensor and a layer thickness sensor disposed on the radially internal side of the tubular member and configured to measure a thickness of the fluid within the airway of the subject in response to a tilting motion of the layer thickness sensor. The layer thickness sensor can be configured to engage in the tilting motion, the viscosity sensor can be configured to engage in the bending motion, or both in response to an application of an external magnetic field. The magnetic actuator can include a magnet coupled to a slider crank mechanism and at least one motor configured to actuate the slider crank mechanism and the magnet. Additionally, one or more components of the device (e.g., the machine-readable instructions, when executed by the processor) can cause the device to at least activate the magnetic actuation device to tilt the layer thickness sensor based at least in part on a first external magnetic field, calculate, using the layer thickness sensor, the thickness of the fluid within the airway of the subject based at least in part on the application of the first external magnetic field, activate the magnetic actuation device to bend the viscosity sensor based at least in part on a second external magnetic field, and calculate, using the viscosity sensor, the viscosity of the fluid within the airway of the subject based at least in part on the application of the second external magnetic field.

In various aspects, the external magnetic field can have a frequency of approximately 0.01 Hz to approximately 10 Hz. In some aspects, the external magnetic field can have a frequency of approximately 0.5 Hz to approximately 8 Hz. In a preferred aspect, the external magnetic field can have a frequency of approximately 0.1 Hz to approximately 5 Hz. In various aspects, the external magnetic field can have a magnitude of approximately 1 mT to approximately 100 mT. In some aspects, the external magnetic field can have a magnitude of approximately 2 mT to approximately 80 mT. In a preferred aspect, the external magnetic field can have a magnitude of approximately 5 mT to approximately 70 mT. In various aspects, the first external magnetic field can have a different frequency, a different magnitude, or both from the second external magnetic field.

In various aspects, the viscosity sensor can include a strain-gauge configured to measure a deformation of the viscosity sensor based at least in part on an electrical resistance of the strain-gauge, where the viscosity of the fluid is calculated based at least in part on the deformation. In various aspects, the layer thickness sensor can include a capacitor configured to measure a capacitance of the capacitor, where the thickness of the fluid is calculated based at least in part on the capacitance. In some aspects, the implant can include at least one rechargeable battery disposed on the radially internal side of the tubular member, wherein the application of the external magnetic field charges the at least one rechargeable battery.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1I show an overview of the sensory artificial cilia for monitoring the conditions of airway stents. FIG. 1A shows a concept of a device for monitoring airway mucus conditions inside a human trachea actuated by external magnetic fields according to various embodiments of the present disclosure. FIG. 1B shows an illustration of the viscosity sensor for sensing liquid viscosity. FIG. 1C shows an illustration of sensor signal outputs for liquids of different viscosities. FIG. 1D shows an illustration of the layer thickness sensor for sensing liquid layer thickness. FIG. 1E shows an illustration of system electronic components and connections. FIG. 1F shows an optical image of the electronic components of the sensory ring. The components can include a Bluetooth Low Energy System-on-a-Chip, two coin-batteries, a viscosity sensor, a layer thickness sensor, and a magnetic sensor (with a temperature sensor inside). FIG. 1G shows an optical image of the electronic components of the sensory ring with a flexible back-layer made of Thermoplastic polyurethane (TPU). FIG. 1H shows the data flow chart of the electronic system and illustration of the user interface. FIG. 1I shows an illustration of a sensory ring and a hybrid airway stent integrated together for sensing mucus properties and the user interface.

FIGS. 2A-2J show the design, fabrication, and calibration of the sensory artificial cilium for sensing liquid viscosity. FIG. 2A shows optical images of the sensory artificial cilium for sensing liquid viscosity, including (i) Overall dimension; and (ii-iii) Zoomed-in optical images of the conductive material before coating (ii) and after coating (iii). FIG. 2B shows schematics of the mechanism of sensing liquid viscosity, including (i) Power stroke; and (ii) Recovery stroke. FIG. 2C shows the resistance of the sensory artificial cilium and its curvature as a function of time when a rotating magnetic field is applied. FIG. 2D shows δR/R₀ of the sensory artificial cilium as a function of time in liquids of different viscosities. δR=R−R₀. R₀: the resistance of the sensory artificial cilium when no magnetic field is applied. FIG. 2E shows the envelope of the time-varying sensor shapes in liquids of different viscosities. In FIGS. 2D-E, magnetic field: f=2 Hz, B=20 mT. FIG. 2F shows δR/R₀ of the viscosity sensor as a function of time in liquids of different viscosities. FIG. 2G shows the envelope of the time-varying sensor shapes in liquids of different viscosities. In FIGS. 2F-G, magnetic field: f=0.2 Hz, B=20 mT. FIG. 2H shows a training dataset and a testing dataset based on measured viscosities and their corresponding the peak-to-peak value of δR/R₀ and f. B=20 mT. Fitting method: Interpolation (see “Materials and Methods”). FIG. 2I shows the measured liquid viscosity as a function of the δR/R₀ at different f. FIG. 2J shows the predicted viscosities of liquids by the calibrated model in FIG. 2H as a function of their measured viscosities.

FIGS. 3A-3F show a characterization of the sensory artificial cilium for mucus viscosity sensing. FIG. 3A shows the measured liquid viscosity as a function of the viscosity sensor resistance pp (δR/R₀) and the magnetic field frequency. FIG. 3B shows the predicted liquid viscosity by the viscosity sensor as a function of the measured liquid viscosity when rotating magnetic fields of different magnitudes are applied. Magnetic field: f=0.2 Hz. Error: [0.03, 0.13, 0.02, 0.07, 0.32, 1.19] Pa·s. FIG. 3C shows the predicted liquid viscosity by the viscosity sensor as a function of the measured liquid viscosity when rotating magnetic fields in different rotating planes are applied. α is the angle between the rotating plane and the x-z plane. Magnetic field: f=0.2 Hz, B=20 mT. Error: [0.14, 0.13, 0.09, 0.27, 0.14, 1.01] Pa·s. FIG. 3D shows the predicted and measured liquid viscosities as a function of the shear rate. The liquid used is porcine mucus prepared by mixing mucin and water with a weight ratio of 1 by 7 and 1 by 8. FIG. 3E shows the time-varying sensor resistance when diluting the mucus with water and heating up the mucus to lose water. FIG. 3F shows optical images of the viscosity sensor inside mucus when sensing the time-varying mucus property.

FIGS. 4A-4I show a characterization of the sensory artificial cilium for liquid layer thickness sensing with reconfigurable sensitivity and range. FIG. 4A shows an optical image of the layer thickness sensor. FIG. 4B shows an optical image of a capacitor-based liquid layer thickness sensor when placed in porcine mucus. FIG. 4C shows the sensor capacitance as a function of mucus layer thickness. FIG. 4D shows the sensor capacitance as a function of the measured thickness of the porcine mucus when varying the mucin concentration in the porcine mucus. FIG. 4E shows an illustration of the calibration process: i. Before submerging; ii. Fully submerging; iii. Measuring mucus thickness.

$${k}_{1} = \frac{{L}_{c} sin {α}_{c}}{{C}_{max} - {C}_{min}} , {h}_{0} = \frac{- {L}_{c} sin {α}_{c}}{{C}_{max} - {C}_{min}} {C}_{min} .$$

FIG. 4F shows the sensor capacitance as a function of time during an online calibration process, dynamic viscosity: 3.5 Pa·s (weight ratio of mucin vs. water: 1 by 9). The calibration procedures include fully submerging the sensor for a maximum capacitor value, allowing the mucus to flow back, and sensing mucus thickness. FIG. 4G shows the measured mucus layer thickness as a function of time using the calibrated model. FIG. 4H shows the sensor capacitance as a function of the measured thickness when controlling the angle of the sensor by external magnetic fields. Magnetic field, B=25 mT. FIG. 4I shows optical images of the mucus layer thickness sensor at different tilting angles. Scale bar, 3 mm. In FIG. 4H and FIG. 4I, the coating polymer is Ecoflex 00-30.

FIGS. 5A-5F show the design and control of a wearable magnetic actuation system for sensory artificial cilia. FIG. 5A is a digital rendering of the front view of a magnetic actuation system. The on-board magnet is rotationally, and translationally actuated by a DC motor and a servo-motor-slider-crank mechanism, respectively. Both actuation modes are controlled by the control electronics board, which is composed of a DC motor driver and a microcontroller. FIG. 5B shows an optical image of the wearable magnetic actuation system with on-board components mounted on a human chest model. FIG. 5C shows an illustration of the magnet relative to the longitudinal axis of a human trachea model and the characterized magnetic field B_{yz} at different locations. FIG. 5D shows the time-varying magnetic field at a location with d_{z}=35 mm and d_{y}=0. FIG. 5E shows an optical image of the viscosity and layer thickness sensors bending or tilting when applying a rotating magnetic field. B=25 mT. FIG. 5F shows modulation of the magnitude and frequency of B_{yz} by controlling the position and angular velocity of the on-board magnet.

FIGS. 6A-6L show a demonstration of the deployment and sensing function of the sensory artificial cilia inside a trachea phantom and a sheep trachea ex vivo. FIG. 6A shows an optical image of the delivery tool with a customized head for constraining the sensory ring. FIG. 6B shows an optical image of the sensory ring inside the head of the delivery tool. FIG. 6C shows video frames of the delivery tool with the sensory ring embedded in the deployment process. FIG. 6D shows video frames of the device deployed inside a trachea phantom: (i) No mucus; (ii) Add mucus, no actuation; (iii) Adding mucus, magnetically actuated; (iv) Added mucus, magnetically actuated. Scale bar, 5 mm. FIG. 6E shows an image of the hybrid stent integrated with the sensory ring. Scale bar, 10 mm. FIG. 6F shows a silicone airway stent integrated with the sensory ring. Scale bar, 10 mm. FIG. 6G shows a real-time viscosity sensor signal when varying the mucus viscosity. FIG. 6H shows a real-time mucus layer thickness sensor signal when varying the mucus layer thickness. When mucus layer thickness passes the threshold, alarm will be triggered for further intervention. FIG. 6I shows an optical image of the sensory ring with a hybrid stent delivered inside a sheep trachea. FIGS. 6J-6L show x-ray images of the sensory ring with a metal stent, only a sensor ring, and a sensor ring with a silicone stent inside a sheep trachea, respectively.

FIG. 7 shows a fabrication process of the viscosity sensor, which combines molding and laser machining. Firstly, in Step 1, graphene is induced on a thin layer of Polyimide (PI) tape using a CO2 laser. Secondly, in Step 2, a thin layer of magnetic composite made of PDMS and NdFeB is coated onto the graphene using a spin coater. Although PDMS is used in FIG. 7, other materials with similar properties and biocompatibility can be used. For example, other silicone elastomers, thermoplastic elastomers (TPEs), poly(methyl methacrylate) (PMMA), polyethylene glycol (PEG), silicone rubber (e.g., Ecoflex™), or other polymer materials can be used. In addition, other materials can be used instead of NdFeB that have similar properties. For example, ferrite, aluminum nickel cobalt (AlNiCo), samarium cobalt (SmCo), iron nitride (FeN), or other magnetic materials can be used. After the magnetic composite is cured, the graphene is tightly bonded to it and then transferred from the PI layer. Subsequently, in Step 3, the heating process facilitates the release of the graphene from the PI layer for easier transfer. In Step 4, the transferred LIG along with the magnetic composite is processed with a UV laser to achieve the desired size and pattern. The pattern is designed as a strain gauge to capture deformation. In Step 5, a small magnetic module is added to the tip of the viscosity sensor to induce a relatively large bending torque. Finally, in Step 6, the viscosity sensor is magnetized and attached to the circuit, with connections made using silver paste. The sensor has a length of 1.75 mm and a width of 1.5 mm with all conductive parts encapsulated with Ecoflex 00-30 to prevent resistance changes due to mucus with different mucin concentrations.

FIGS. 8A-8C show optical images of the viscosity sensor. FIG. 8A shows an image of LIG after laser patterning. FIG. 8B shows a microscopic image of the LIG pattern. FIG. 8C shows an optical image of a viscosity sensor in a side view with marked dimensions.

FIGS. 9A and 9B show a comparison of viscosity sensors with and without encapsulation inside mucus. FIG. 9A shows a sensor signal of a sensor with encapsulation. FIG. 9B shows a sensor signal of a sensor without encapsulation. In the legends, “m:w” indicates the weight ratio between mucin and water.

FIG. 10 shows an experimental setup for testing sensing mucus viscosity and layer thickness.

FIGS. 11A and 11B show an illustration of the sensory cilium for viscosity sensing. FIG. 11A shows a free-body diagram of the sensory cilium inside a liquid. FIG. 11B shows an illustration of the length change of the LIG layer.

FIGS. 12A and 12B show a modeling of the viscosity sensor. FIG. 12A shows a sensor shape when placed in liquids of different viscosities. FIG. 12B shows a sensor shape when being actuated by magnetic fields of different frequences. μ₀=1 Pa·s.

FIG. 13 shows a viscosity sensor resistance as a function of time when applying magnetic fields of different magnitudes.

FIG. 14 shows a fabrication process of the layer thickness sensors.

FIGS. 15A and 15B show an illustration of the mechanism of the mucus layer thickness sensor. FIG. 15A shows a perspective view of the layer thickness sensor. FIG. 15B shows a cross-section view of the capacitor sensor with covered mucus.

FIGS. 16A and 16B show a modeling of the layer thickness sensor using the fringe-effect capacitor. FIG. 16A shows a distribution of the electric field. FIG. 16B shows a simulation of the capacitance as a function of the liquid layer thickness.

FIGS. 17A and 17B show an optical image of the layer thickness sensor with marked dimensions. FIG. 17A shows optical images of the PDMS-coated and uncoated layer thickness sensors. FIG. 17B shows an optical image of the marked dimensions of the magnetic layer thickness.

FIG. 18 shows capacitance of a mucus layer thickness sensor as a function of the mucus layer thickness without polymer encapsulation.

FIGS. 19A-D show a wirelessly controlled wearable magnetic actuation system. FIG. 19A shows an illustration of the wearable magnetic actuation system. FIG. 19B shows an optical image of the wearable magnetic actuation system controlled by a cell phone via Bluetooth. FIG. 19C shows an optical image of a sensor ring in a trachea phantom. FIG. 19D shows an image of the data plots in a software ‘Bluefruit LE Connect’ on a cell phone when receiving data from the sensors.

FIGS. 20A-F show magnetic field angle differences of the wearable magnetic actuation system in different orientations, captured by a magnetic sensor. FIG. 20A shows an initial orientation of the wearable magnetic actuation system, described by Frame 0. FIG. 20B shows a second orientation of the wearable magnetic actuation system, described by Frame 1. FIG. 20C shows a measured magnetic field generated by the wearable magnetic actuation system in its initial orientation. FIG. 20D shows a measured magnetic field generated by the wearable magnetic actuation system in its second orientation. FIG. 20E shows the calculated normal vector of the magnetic field plane from results in FIG. 20C denoted by no. FIG. 20F shows the calculated normal vector of the magnetic field plane from results in FIG. 20D, denoted by n₁.

FIGS. 21A and 21B show a mapping between the tilting angle of the mucus layer thickness sensor and the magnetic field angle. FIG. 21A shows an illustration of the tilting angle of the mucus layer thickness sensor and the magnetic field orientations. The tilting angle of the mucus layer thickness sensor is calculated by selecting points on the sensor bottom, tip, and the horizontal plane, shown in red circles. FIG. 21B shows a relationship of the tilting angle of the mucus layer thickness sensor and the magnetic field orientations. Magnetic field magnitude: 25 mT.

FIGS. 22A-C show a resilience of the sensory ring during the deployment process. FIG. 22A shows sequential images of deploying a hybrid stent with the sensory ring. FIG. 22B shows screenshots of the sensor signal plots before and after the deployment. FIG. 22C shows sequential images of deploying a silicone stent with the sensory ring. Scale bars, 5 mm.

FIGS. 23A and 23B show characterization of the variations of the mucus viscosity sensors. FIG. 23A shows δR/R₀ of three separately prepared viscosity sensors as a function of time when actuated in syrup. Liquid viscosity: 9.6 Pa·s. Magnetic field frequency: 0.5 Hz. Magnetic field magnitude: 20 mT. FIG. 23B shows optical images of three separately prepared viscosity sensors in the side and top views.

FIGS. 24A and 24B show characterization of the variations of the layer thickness sensors. FIG. 24A shows the capacitance of three separately prepared layer thickness sensors as a function of the mucus layer thickness sensor. Mucus to water ratio is 1 to 8. FIG. 24B shows capacitance of a sensor as a function of time when adding mucus to increase the layer thickness.

FIGS. 25A and 25B show a characterization of the durability of a viscosity sensor. FIG. 25A shows sensor resistance as a function of the beating cycle when actuated by magnetic fields in syrup. FIG. 25B shows zoomed-in plots of the sensor resistance as a function of the beating cycle. Magnetic field frequency: 1 Hz. Magnetic field magnitude: 20 mT. Liquid viscosity: 9.2 Pa·s.

FIG. 26 shows a characterization of the durability of a layer thickness sensor. Sensor capacitance is shown as a function of the bending cycle when actuated by magnetic fields in mucus.

FIGS. 27A and 27B show a battery voltage as a function of time when continuously operating in different modes. FIG. 27A shows the voltage of two batteries (LR626, 1.5 Volts, 18 mAh) when the BLE SoC is in a working mode. Magnetic sensor: low power mode. Loop update rate: 5 Hz. FIG. 27B shows battery voltage when the BLE Soc is in a sleeping mode. As the power consumption in the sleeping mode is negligible compared to the working mode, the device's lifespan is estimated using the operational period in FIG. 27A divided by ratio of the working mode time (10 seconds) over a full operation cycle duration (30 minutes).

FIG. 28 shows circuit schematics of the fully integrated sensor. The mucus viscosity sensor is connected to LIG1 and AIN1 (Analog Input 1). The mucus layer thickness sensor is connected to AIN1 (Analog Input 2) and AIN6 (Analog Input 6).

FIG. 29 shows a parameter of the surface fitting function for the viscosity sensor.

FIG. 30 shows a list of the components of the sensory ring.

FIG. 31 shows technical parameters of the sensor with integration of the circuit.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth to provide those of ordinary skill in the art with a complete disclosure and description of how to perform the methods and use the compositions and compounds disclosed and claimed herein. Efforts have been made to ensure accuracy with respect to numbers (e.g., amounts, measurements, etc.), but some errors and deviations should be accounted for.

Before the embodiments of the present disclosure are described in detail, it is to be understood that, unless otherwise indicated, the present disclosure is not limited to particular materials, machines, computing processes, or the like, as such can vary. It is also to be understood that the terminology used herein is for purposes of describing particular embodiments only and is not intended to be limiting. It is also possible in the present disclosure that steps can be executed in different sequence where this is logically possible.

It should be noted that ratios, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

### DISCUSSION

Disclosed are various approaches for a sensory artificial cilia for in situ monitoring of airway physiological properties. Continuously monitoring human airway conditions can be important for timely interventions, especially when airway stents are implanted to alleviate central airway obstruction in lung cancer and other diseases. Mucus conditions can be important biomarkers for indicating inflammation and stent patency inside the human airway but remain challenging to monitor. Current methods, such as computational tomography imaging and bronchoscope inspection, pose risks due to radiation exposure and often lack the ability to provide continuous real-time feedback outside of hospital settings.

Accordingly, various embodiments of the present disclosure are directed to systems and methods for using a sensory artificial cilia for in situ monitoring of airway physiological properties. Inspired by the sensing ability of biological cilia, the present disclosure provides for wireless sensing mechanisms in sensory artificial cilia for detecting mucus conditions, including viscosity and layer thickness, which are crucial biomarkers for disease severity. The sensing mechanisms in artificial cilia described herein can allow continuous monitoring of the properties of biofluids inside the airway and can be integrated with implantable devices, such as airway stents, to monitor various fluidic conditions for disease monitoring, enabling timely interventions. The sensing mechanism for mucus viscosity leverages external magnetic fields to actuate a magnetic artificial cilium and sense its shape using a flexible strain gauge. Additionally, the present disclosure provides an artificial cilium with capacitance sensing for mucus layer thickness, offering unique self-calibration, adjustable sensitivity, and range, all enabled by external magnetic fields. To enable prolonged and wireless data access, wireless communication and onboard power can be integrated, along with a wearable magnetic actuation system for sensor activation. The artificial cilium sensor can be deployed independently or in conjunction with an airway stent. The sensing mechanisms and devices of the present disclosure can pave the way for real-time monitoring of mucus conditions, facilitating early disease detection and providing stent patency alerts, thereby allowing timely interventions and personalized care.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

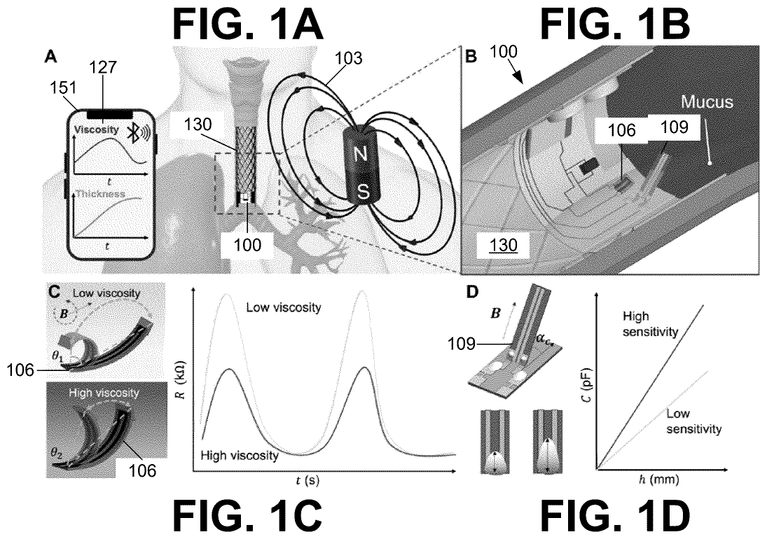

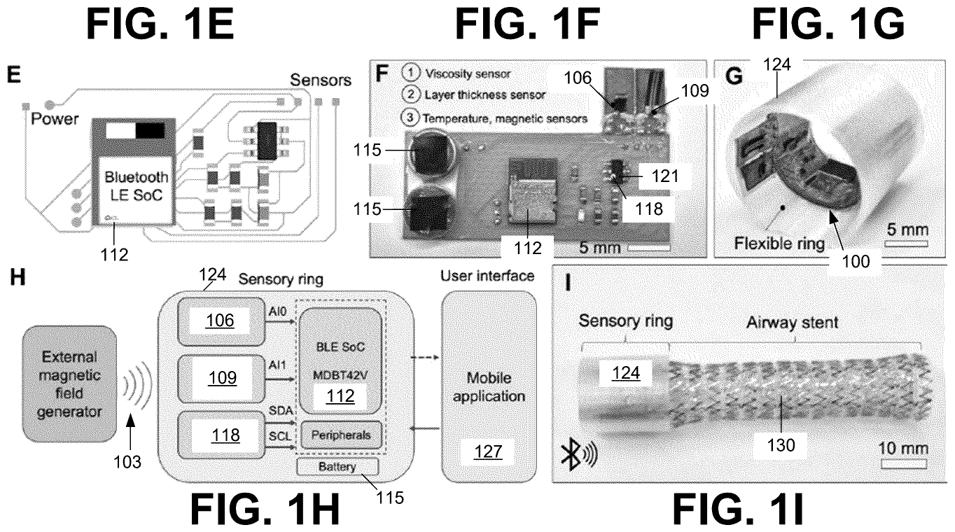

With reference to FIGS. 1A-1I, shown is an example concept of the sensory artificial cilia for monitoring the conditions of airway stents. FIG. 1A shows a concept of a device 100 for monitoring airway mucus conditions inside a human trachea actuated by external magnetic fields 103 according to various embodiments of the present disclosure. FIG. 1B shows an illustration of the viscosity sensor 106 for sensing liquid viscosity. FIG. 1C shows an illustration of sensor signal outputs for liquids of different viscosities. FIG. 1D shows an illustration of the layer thickness sensor 109 for sensing liquid layer thickness. FIG. 1E shows an illustration of system electronic components and connections. FIG. 1F shows an optical image of the electronic components of the sensory ring. The components can include a Bluetooth Low Energy System-on-a-Chip 112, two coin-batteries 115, a viscosity sensor 106, a layer thickness sensor 109, and a magnetic sensor 118 (with a temperature sensor 121 inside). FIG. 1G shows an optical image of the electronic components of the sensory ring 124 with a flexible back-layer made of Thermoplastic polyurethane (TPU). FIG. 1H shows the data flow chart of the electronic system and illustration of the user interface 127. FIG. 1I shows an illustration of a sensory ring 124 and a hybrid airway stent 130 integrated together for sensing mucus properties and the user interface 127.

As shown in FIGS. 1A and 1B, the device 100 can include a tubular member (e.g., a sensory ring 124, a stent 130, etc.) having a radially external side and a radially internal side, representing the inside surface and the outside surface of the tubular member. The tubular member can be silicone, silicone rubber, elastomer, other soft materials, or a metal for placement in an airway of a subject. The device 100 can include a viscosity sensor 106 configured to measure a viscosity of a fluid within an airway of a subject in response to a bending motion of the viscosity sensor 106 (FIG. 1C) and a layer thickness sensor 109 configured to measure a thickness of the fluid within the airway of the subject in response to a tilting motion of the layer thickness sensor 109 (FIG. 1D). The layer thickness sensor 109 can be configured to engage in the tilting motion, the viscosity sensor 106 can be configured to engage in the bending motion, or both in response to an application of an external magnetic field 103, as shown in FIGS. 1A, 1C, and 1D. In various aspects, the device 100 can be configured to wirelessly communicate with one or more computing devices 251 (FIG. 1A) to display data from the sensors on a user interface 127. In some aspects, the device can include an airway stent 130 coupled to one or more ends of the device 100. As shown in FIG. 1E, FIG. 1F, and FIG. 1H, for example, the device 100 can include a wireless communication module 112 configured to wirelessly communicate with one or more computing devices 251, as well as one or more batteries 115, a viscosity sensor 106, a layer thickness sensor 109, a temperature sensor 121, and/or one or more magnetic sensors 118. In some aspects, the tubular member of the device 100 can be a sensory ring 124. The sensory ring 124 can include a flexible material, such as thermoplastic polyurethane (TPU) (FIG. 1G). However, the tubular member of the device 100 can include other polymers with similar properties to TPU, such as thermoplastic elastomers (TPEs), thermoplastic copolyesters (TPC), silicone rubbers, or other polymer materials that are biocompatible. As shown in FIG. 1I, the device 100 can be attached to an airway stent 130 at one end.

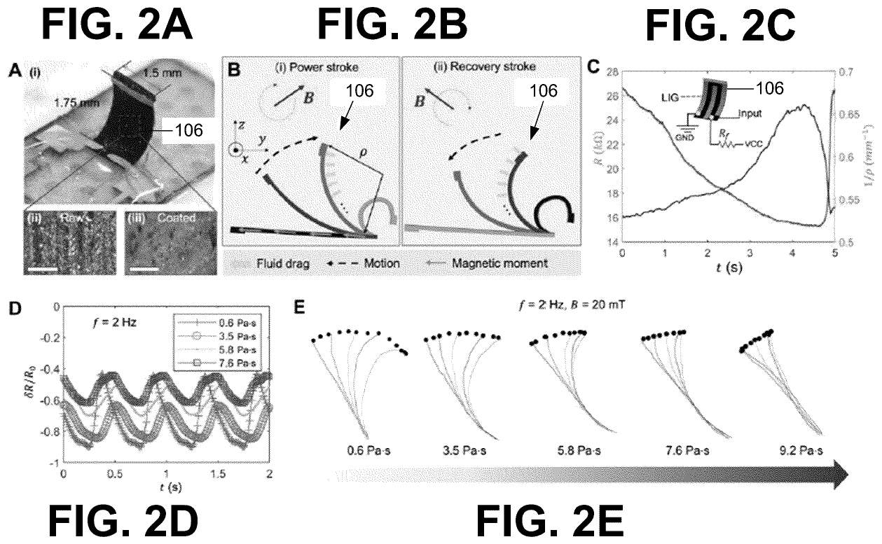

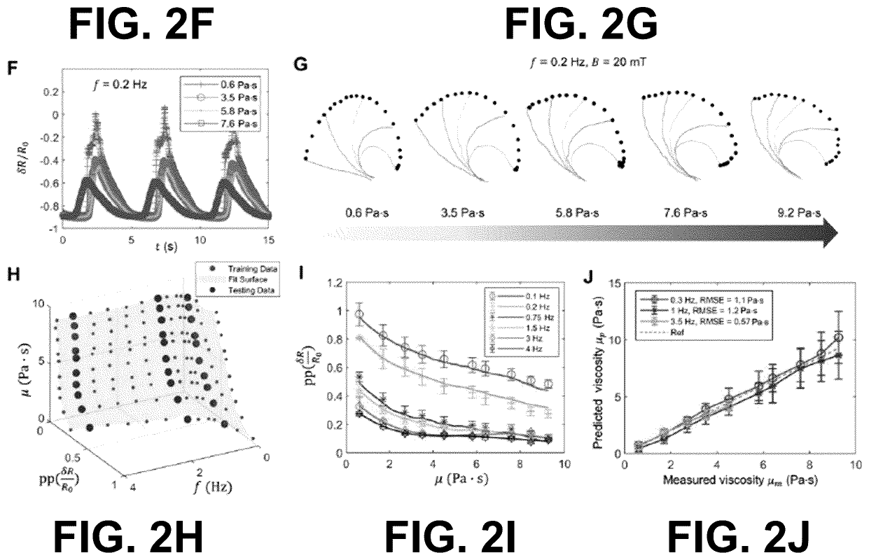

Moving on to FIGS. 2A-2J, shown is the design, fabrication, and calibration of the sensory artificial cilium for sensing liquid viscosity. FIG. 2A shows optical images of the sensory artificial cilium for sensing liquid viscosity, including (i) Overall dimension; and (ii-iii) Zoomed-in optical images of the conductive material before coating (ii) and after coating (iii). In various aspects, the viscosity sensor 106 can have a length of approximately 0.01 mm to approximately 3 mm. In some aspects, the viscosity sensor 106 can have a length of approximately 1 mm to approximately 2 mm. As shown in FIG. 2A, in some aspects the viscosity sensor 106 can have a length of approximately 1.75 mm. In various aspects, the viscosity sensor 106 can have a width of approximately 0.01 mm to approximately 3 mm. In some aspects, the viscosity sensor 106 can have a width of approximately 1 mm to approximately 2 mm. As shown in FIG. 2A, in some aspects the viscosity sensor 106 can have a width of approximately 1.5 mm. FIG. 2B shows schematics of the mechanism of sensing liquid viscosity, including (i) Power stroke; and (ii) Recovery stroke. FIG. 2C shows the resistance of the sensory artificial cilium and its curvature as a function of time when a rotating magnetic field 103 is applied. FIG. 2D shows δR/R₀ of the sensory artificial cilium as a function of time in liquids of different viscosities. δR=R−R₀. R₀: the resistance of the sensory artificial cilium when no magnetic field 103 is applied. FIG. 2E shows the envelope of the time-varying sensor shapes in liquids of different viscosities. In FIGS. 2D-E, magnetic field 103: f=2 Hz, B=20 mT. FIG. 2F shows δR/R₀ of the viscosity sensor 106 as a function of time in liquids of different viscosities. FIG. 2G shows the envelope of the time-varying sensor shapes in liquids of different viscosities. In FIGS. 2F-G, magnetic field 103: f=0.2 Hz, B=20 mT. FIG. 2H shows a training dataset and a testing dataset based on measured viscosities and their corresponding the peak-to-peak value of δR/R₀ and f. B=20 mT. Fitting method: Interpolation (see “Materials and Methods”). FIG. 2I shows the measured liquid viscosity as a function of the δR/R₀ at different f. FIG. 2J shows the predicted viscosities of liquids by the calibrated model in FIG. 2H as a function of their measured viscosities.

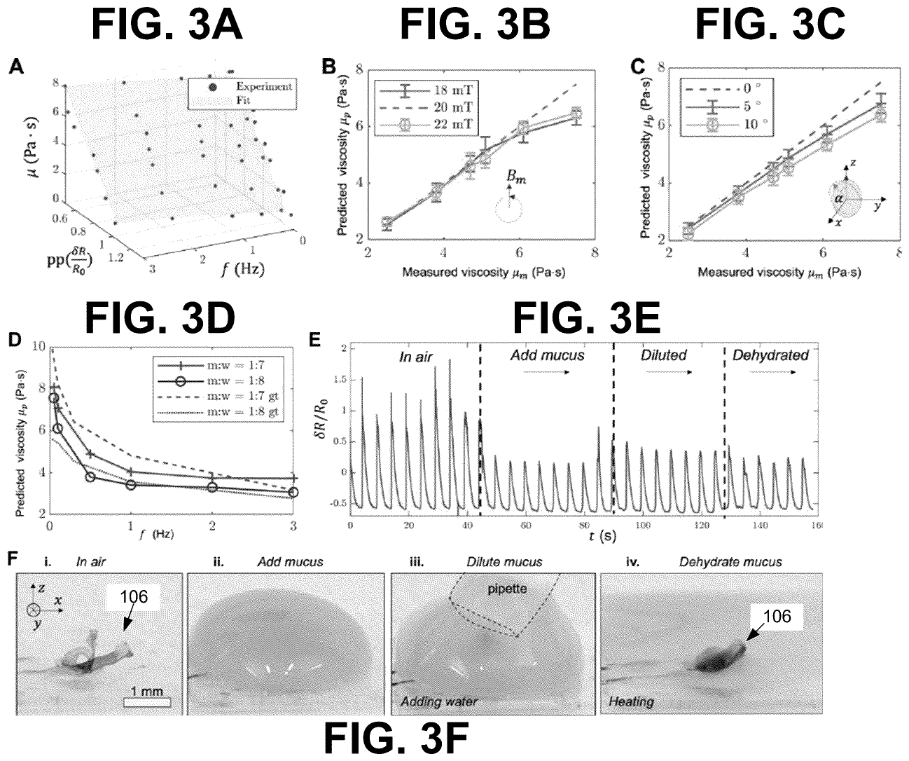

Turning now to FIGS. 3A-3F, shown is an example characterization of the sensory artificial cilium for mucus viscosity sensing. FIG. 3A shows the measured liquid viscosity as a function of the viscosity sensor 106 resistance pp (δR/R₀) and the magnetic field 103 frequency. FIG. 3B shows the predicted liquid viscosity by the viscosity sensor 106 as a function of the measured liquid viscosity when rotating magnetic fields 103 of different magnitudes are applied. Magnetic field 103: f=0.2 Hz. Error: [0.03, 0.13, 0.02, 0.07, 0.32, 1.19] Pa·s. FIG. 3C shows the predicted liquid viscosity by the viscosity sensor 106 as a function of the measured liquid viscosity when rotating magnetic fields 103 in different rotating planes are applied. α is the angle between the rotating plane and the x-z plane. Magnetic field 103: f=0.2 Hz, B=20 mT. Error: [0.14, 0.13, 0.09, 0.27, 0.14, 1.01] Pa·s. FIG. 3D shows the predicted and measured liquid viscosities as a function of the shear rate. The liquid used is porcine mucus prepared by mixing mucin and water with a weight ratio of 1 by 7 and 1 by 8. FIG. 3E shows the time-varying sensor resistance when diluting the mucus with water and heating up the mucus to lose water. FIG. 3F shows optical images of the viscosity sensor 106 inside mucus when sensing the time-varying mucus property.

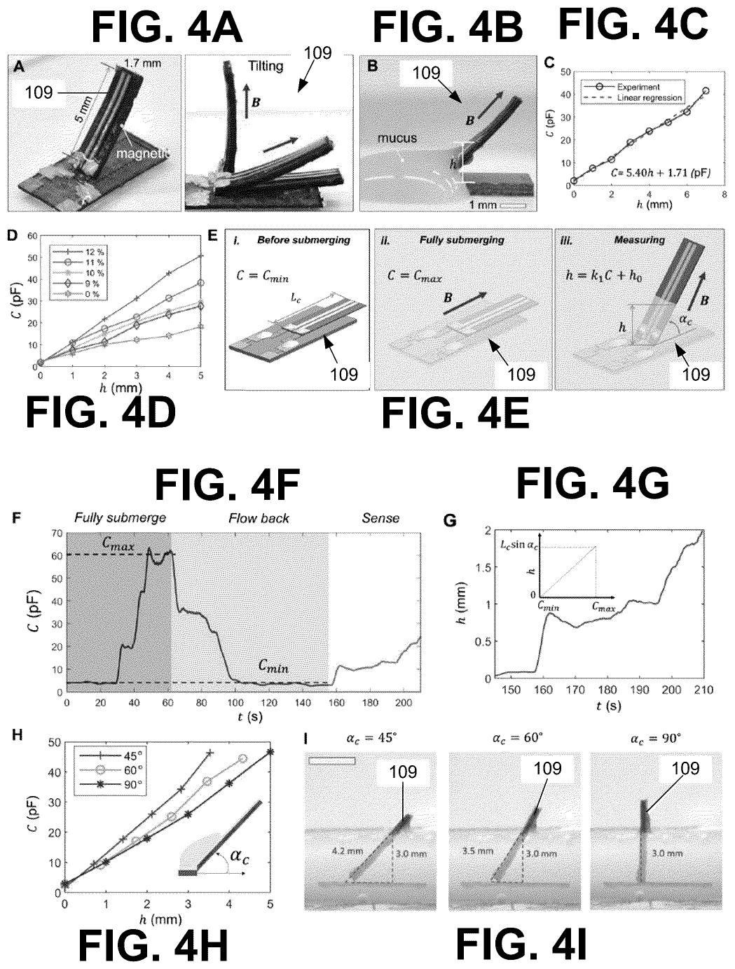

Moving on to FIGS. 4A-41, shown is an example characterization of the sensory artificial cilium for liquid layer thickness sensing with reconfigurable sensitivity and range. FIG. 4A shows an optical image of the layer thickness sensor 109. The layer thickness sensor 109 can be approximately 5 mm long and approximately 1.7 mm wide. However, other sizes are also possible. In various aspects, the layer thickness sensor 109 can have a length of approximately 1 mm to approximately 6 mm. In some aspects, the layer thickness sensor 109 can have a length of approximately 3 mm to approximately 5 mm. In various aspects, the layer thickness sensor 109 can have a width of approximately 1 mm to approximately 5 mm. In some aspects, the layer thickness sensor 109 can have a width of approximately 1.5 mm to approximately 3 mm. FIG. 4B shows an optical image of a capacitor-based liquid layer thickness sensor 109 when placed in porcine mucus. FIG. 4C shows the sensor capacitance as a function of mucus layer thickness. FIG. 4D shows the sensor capacitance as a function of the measured thickness of the porcine mucus when varying the mucin concentration in the porcine mucus. FIG. 4E shows an illustration of the calibration process: i. Before submerging; ii. Fully submerging; iii. Measuring mucus thickness.

$${k}_{1} = \frac{{L}_{c} sin {α}_{c}}{{C}_{max} - {C}_{min}} , {h}_{0} = \frac{- {L}_{c} sin {α}_{c}}{{C}_{max} - {C}_{min}} {C}_{min} .$$

FIG. 4F shows the sensor capacitance as a function of time during an online calibration process, dynamic viscosity: 3.5 Pa·s (weight ratio of mucin vs. water: 1 by 9). The calibration procedures include fully submerging the sensor 109 for a maximum capacitor value, allowing the mucus to flow back, and sensing mucus thickness. FIG. 4G shows the measured mucus layer thickness as a function of time using the calibrated model. FIG. 4H shows the sensor capacitance as a function of the measured thickness when controlling the angle of the sensor by external magnetic fields 103. Magnetic field 103, B=25 mT. FIG. 4I shows optical images of the mucus layer thickness sensor 109 at different tilting angles. Scale bar, 3 mm. In FIG. 4H and FIG. 4I, the coating polymer is Ecoflex 00-30. However, other coating polymers are also possible.

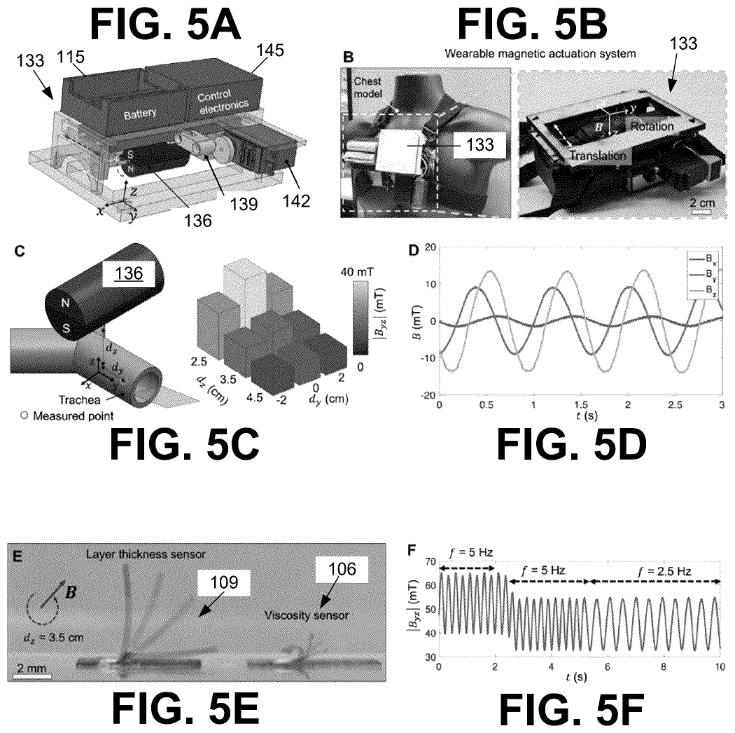

Next, at FIGS. 5A-5F, shown is an example design and control of a wearable magnetic actuation system 133 for sensory artificial cilia. FIG. 5A is a digital rendering of the front view of a magnetic actuation system 133. The on-board magnet 136 is rotationally, and translationally actuated by a DC motor 139 and a servo-motor-slider-crank mechanism 142, respectively. Both actuation modes are controlled by the control electronics board 145, which is composed of a DC motor 139 driver and a microcontroller. FIG. 5B shows an optical image of the wearable magnetic actuation system 133 with on-board components mounted on a human chest model. Although the magnetic actuation system 133 is shown on a chest of a subject, other placements of the magnetic actuation system 133 are possible. The magnetic actuation system 133 can be placed in a variety of locations near the implanted device 100, as long as the magnetic actuation system 133 is configured to actuate the viscosity sensor 106 and the layer thickness sensor 109 of the device 100. FIG. 5C shows an illustration of the magnet 136 relative to the longitudinal axis of a human trachea model and the characterized magnetic field 103 B_{yz} at different locations. FIG. 5D shows the time-varying magnetic field 103 at a location with d_{z}=35 mm and d_{y}=0. FIG. 5E shows an optical image of the viscosity 106 and layer thickness sensors 109 bending or tilting when applying a rotating magnetic field 103. B=25 mT. FIG. 5F shows modulation of the magnitude and frequency of B_{yz} by controlling the position and angular velocity of the on-board magnet 136.

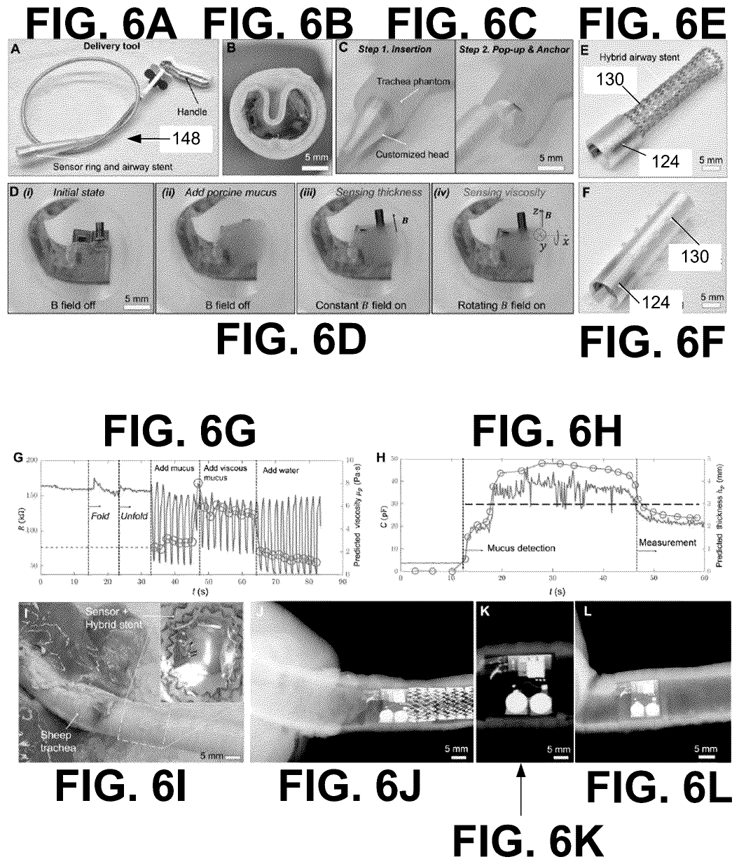

As depicted in FIGS. 6A-6L, shown is a demonstration of the deployment and sensing function of the sensory artificial cilia inside a trachea phantom and a sheep trachea ex vivo. FIG. 6A shows an optical image of the delivery tool 148 with a customized head for constraining the sensory ring 124. FIG. 6B shows an optical image of the sensory ring 124 inside the head of the delivery tool 148. FIG. 6C shows video frames of the delivery tool 148 with the sensory ring 124 embedded in the deployment process. FIG. 6D shows video frames of the device 100 deployed inside a trachea phantom: (i) No mucus; (ii) Add mucus, no actuation; (iii) Adding mucus, magnetically actuated; (iv) Added mucus, magnetically actuated. Scale bar, 5 mm. FIG. 6E shows an image of the hybrid stent 130 integrated with the sensory ring 124. Scale bar, 10 mm. FIG. 6F shows a silicone airway stent 130 integrated with the sensory ring 124. Scale bar, 10 mm. FIG. 6G shows a real-time viscosity sensor 106 signal when varying the mucus viscosity. FIG. 6H shows a real-time mucus layer thickness sensor 109 signal when varying the mucus layer thickness. When mucus layer thickness passes the threshold, an alarm will be triggered for further intervention. FIG. 6I shows an optical image of the sensory ring 124 with a hybrid stent 130 delivered inside a sheep trachea. FIGS. 6J-6L show x-ray images of the sensory ring 124 with a metal stent 130, only a sensor ring 124, and a sensor ring 124 with a silicone stent 130 inside a sheep trachea, respectively.

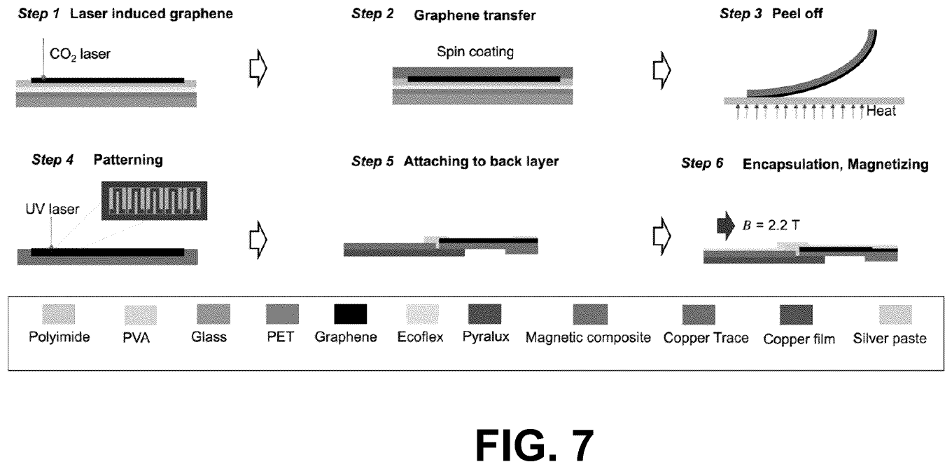

Next, FIG. 7 shows an example fabrication process of the viscosity sensor 106, which combines molding and laser machining. Firstly, in Step 1, graphene is induced on a thin layer of Polyimide (PI) tape using a CO2 laser. Secondly, in Step 2, a thin layer of magnetic composite made of PDMS and NdFeB is coated onto the graphene using a spin coater. After the magnetic composite is cured, the graphene is tightly bonded to it and then transferred from the PI layer. Subsequently, in Step 3, the heating process facilitates the release of the graphene from the PI layer for easier transfer. In Step 4, the transferred LIG along with the magnetic composite is processed with a UV laser to achieve the desired size and pattern. The pattern is designed as a strain gauge to capture deformation. In Step 5, a small magnetic module is added to the tip of the viscosity sensor 106 to induce a relatively large bending torque. Finally, in Step 6, the viscosity sensor 106 is magnetized and attached to the circuit, with connections made using silver paste. In the example of FIG. 7, the sensor 106 has a length of 1.75 mm and a width of 1.5 mm with all conductive parts encapsulated with Ecoflex 00-30 to prevent resistance changes due to mucus with different mucin concentrations. Other dimensions and materials are possible, however.

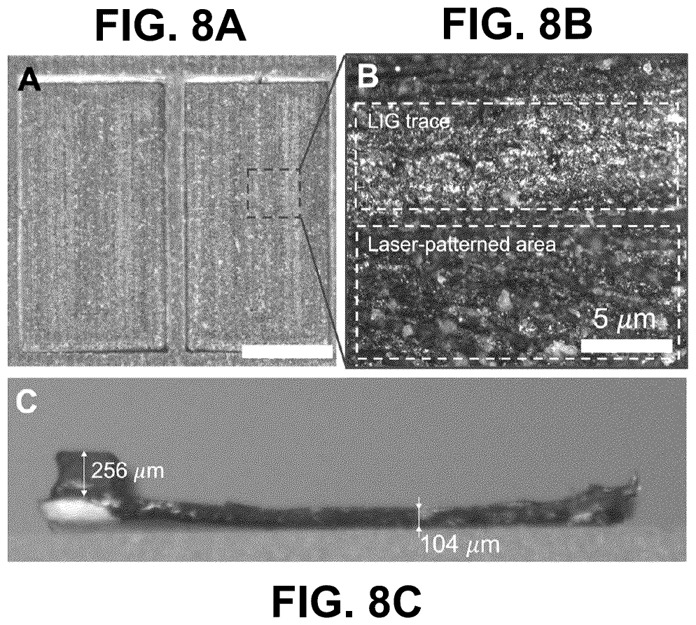

Turning now to FIGS. 8A-8C, shown are optical images of the viscosity sensor 106. FIG. 8A shows an image of LIG after laser patterning. FIG. 8B shows a microscopic image of the LIG pattern. FIG. 8C shows an optical image of a viscosity sensor 106 in a side view with marked dimensions. However, other dimensions are possible.

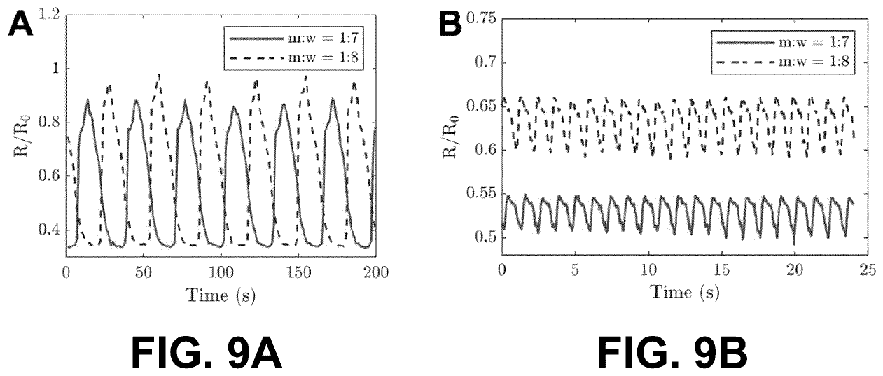

As depicted in FIGS. 9A and 9B, shown is an example comparison of viscosity sensors 106 with and without encapsulation inside mucus. FIG. 9A shows a sensor signal of a sensor 106 with encapsulation. FIG. 9B shows a sensor signal of a sensor 106 without encapsulation. In the legends, “m:w” indicates the weight ratio between mucin and water.

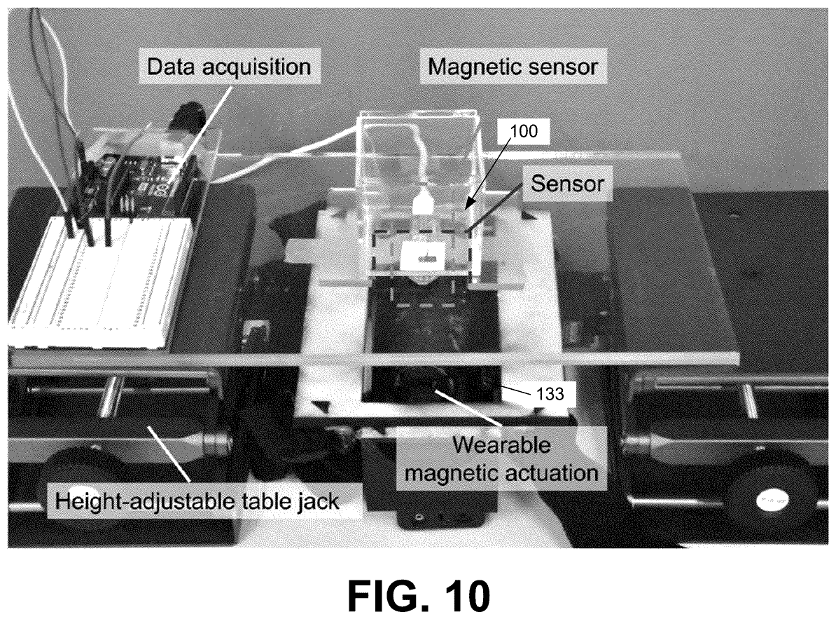

Next, FIG. 10 shows an example experimental setup for testing sensing mucus viscosity and layer thickness.

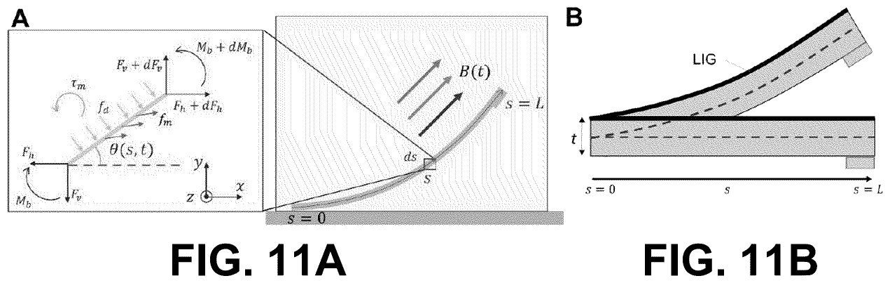

FIGS. 11A and 11B show an example illustration of the sensory cilium for viscosity sensing. FIG. 11A shows a free-body diagram of the sensory cilium inside a liquid. FIG. 11B shows an illustration of the length change of the LIG layer.

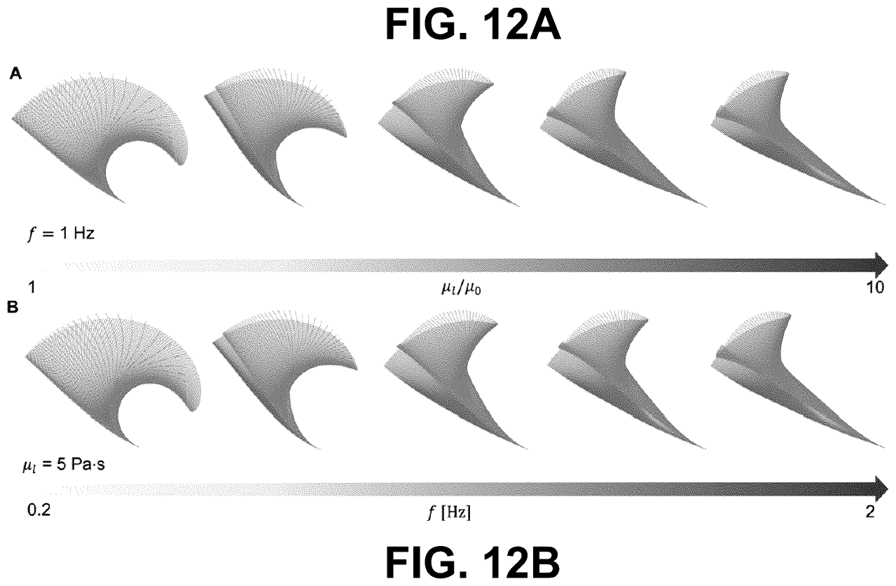

Moving to FIGS. 12A and 12B, shown is a modeling of the viscosity sensor 106. FIG. 12A shows a sensor shape when placed in liquids of different viscosities. FIG. 12B shows a sensor shape when being actuated by magnetic fields 103 of different frequences. μ₀=1 Pa·s.

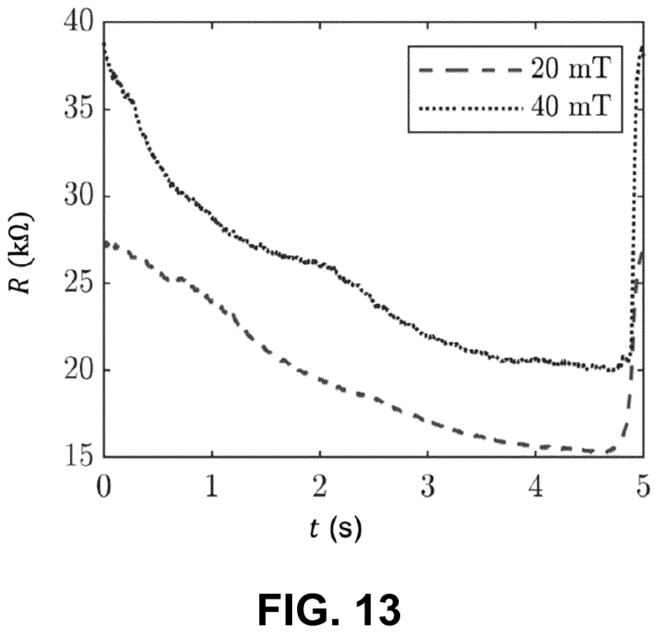

Next, at FIG. 13, shown is a viscosity sensor 106 resistance as a function of time when applying magnetic fields 103 of different magnitudes.

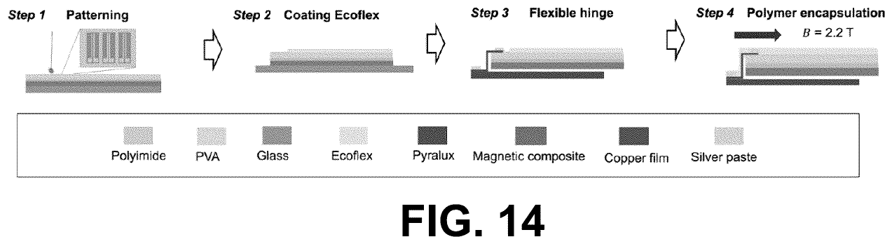

Next, FIG. 14 shows an example fabrication process of the layer thickness sensors 109. Other materials than the ones shown in the example of FIG. 14 are possible.

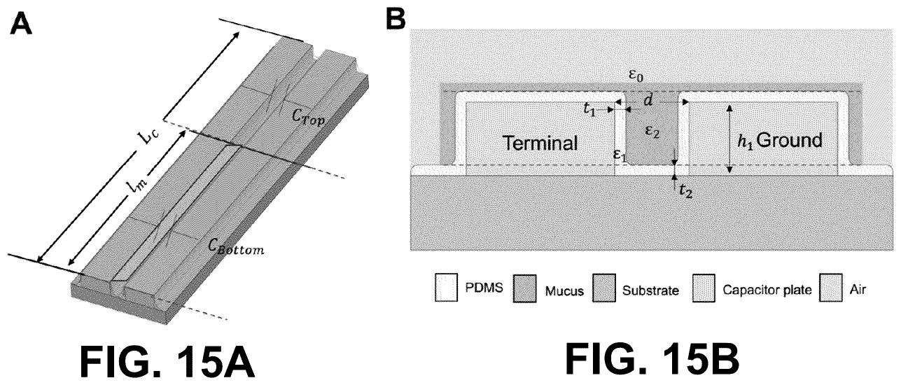

Moving to FIGS. 15A and 15B, shown is an illustration of the mechanism of the mucus layer thickness sensor 109. FIG. 15A shows a perspective view of the layer thickness sensor 109. FIG. 15B shows a cross-section view of the capacitor sensor with covered mucus.

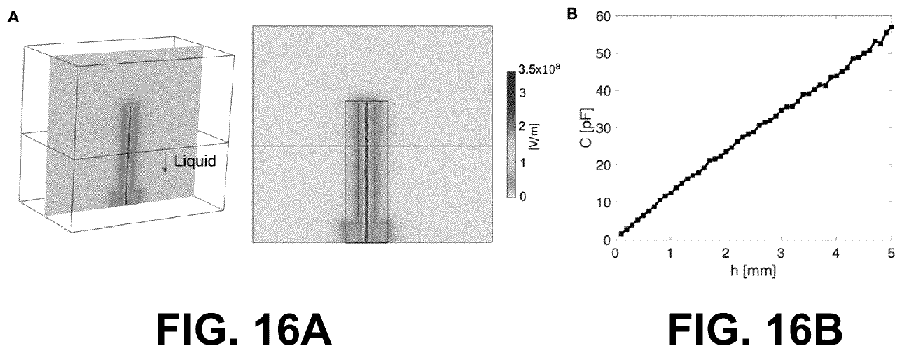

At FIGS. 16A and 16B, shown is a modeling of the layer thickness sensor 109 using the fringe-effect capacitor. FIG. 16A shows a distribution of the electric field. FIG. 16B shows a simulation of the capacitance as a function of the liquid layer thickness.

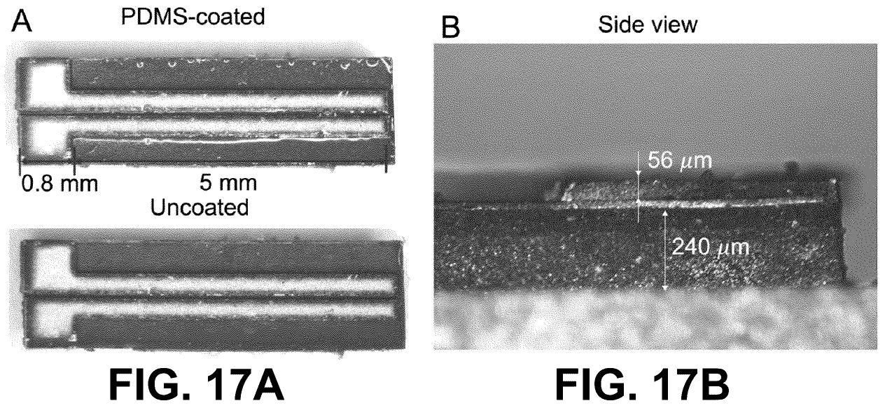

Moving to FIGS. 17A and 17B, shown is an optical image of the layer thickness sensor 109 with marked dimensions. However, other dimensions than the ones shown in FIGS. 17A and 17B are possible. FIG. 17A shows optical images of the PDMS-coated and uncoated layer thickness sensors 109. FIG. 17B shows an optical image of the marked dimensions of the magnetic layer thickness.

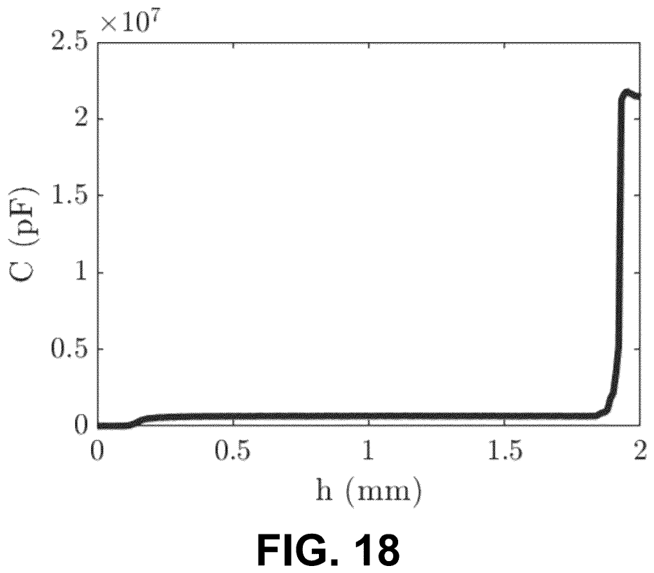

In FIG. 18, shown is an example capacitance of a mucus layer thickness sensor 109 as a function of the mucus layer thickness without polymer encapsulation.

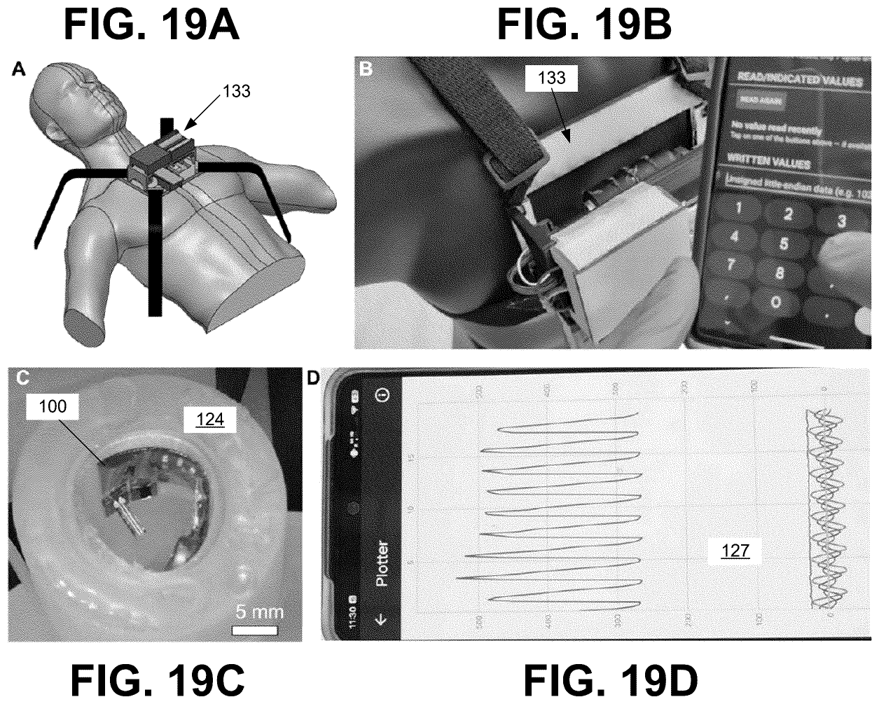

Turning to FIGS. 19A-D, shown is a wirelessly controlled wearable magnetic actuation system 133. FIG. 19A shows an illustration of the wearable magnetic actuation system 133. FIG. 19B shows an optical image of the wearable magnetic actuation system 133 controlled by a cell phone (a computing device 151) via Bluetooth. FIG. 19C shows an optical image of a sensor ring 124 in a trachea phantom. FIG. 19D shows an image of the data plots in a software ‘Bluefruit LE Connect’ on a cell phone when receiving data from the sensors.

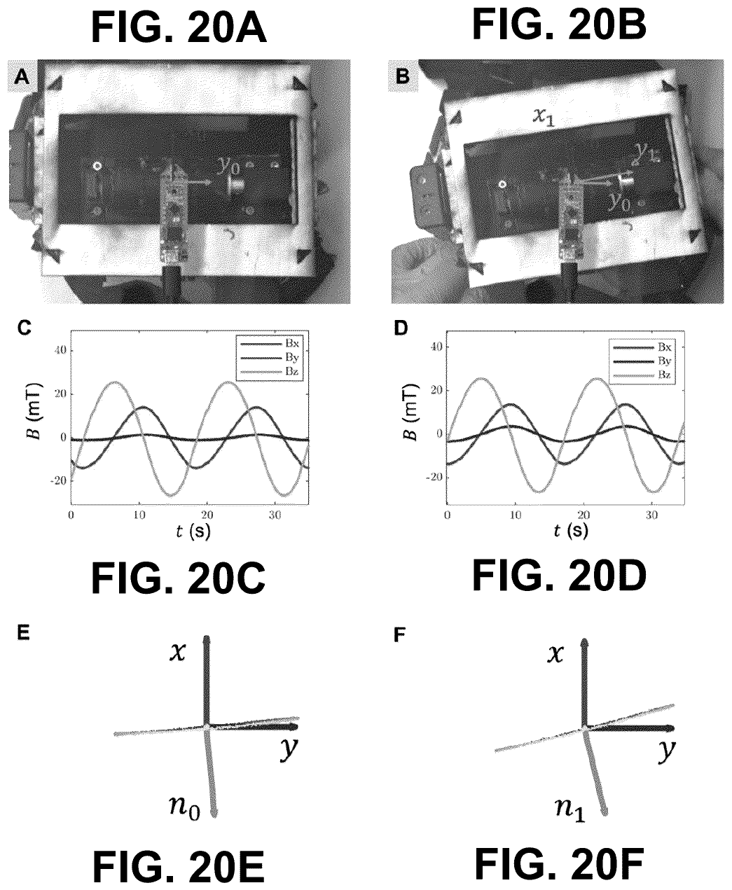

Next, FIGS. 20A-F show magnetic field 103 angle differences of the wearable magnetic actuation system 133 in different orientations, captured by a magnetic sensor 118. FIG. 20A shows an initial orientation of the wearable magnetic actuation system 133, described by Frame 0. FIG. 20B shows a second orientation of the wearable magnetic actuation system 133, described by Frame 1. FIG. 20C shows a measured magnetic field 103 generated by the wearable magnetic actuation system 133 in its initial orientation. FIG. 20D shows a measured magnetic field 103 generated by the wearable magnetic actuation system 133 in its second orientation. FIG. 20E shows the calculated normal vector of the magnetic field 103 plane from results in FIG. 20C denoted by no. FIG. 20F shows the calculated normal vector of the magnetic field 103 plane from results in FIG. 20D, denoted by n₁.

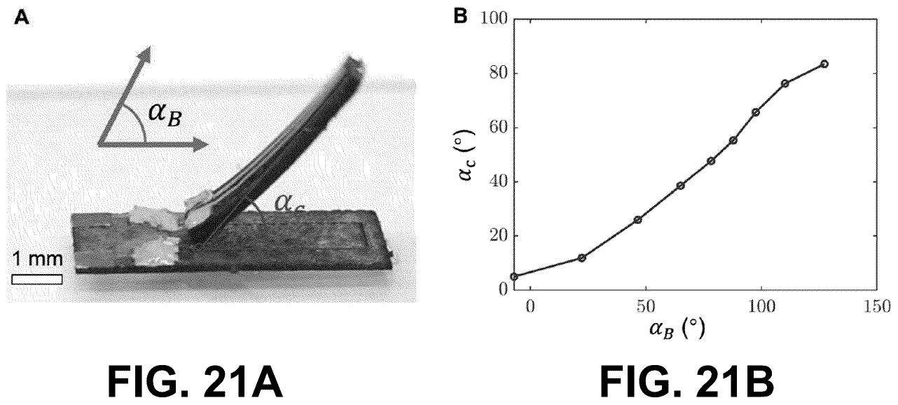

In FIGS. 21A and 21B, shown is a mapping between the tilting angle of the mucus layer thickness sensor 109 and the magnetic field 103 angle. FIG. 21A shows an illustration of the tilting angle of the mucus layer thickness sensor 109 and the magnetic field 103 orientations. The tilting angle of the mucus layer thickness sensor 109 is calculated by selecting points on the sensor bottom, tip, and the horizontal plane, shown in red circles. FIG. 21B shows a relationship of the tilting angle of the mucus layer thickness sensor 109 and the magnetic field 103 orientations. Magnetic field 103 magnitude: 25 mT.

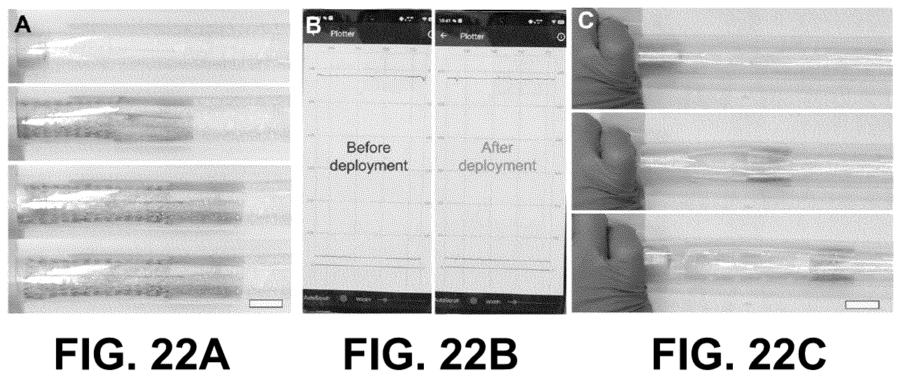

FIGS. 22A-C show a resilience of the sensory ring 124 during the deployment process. FIG. 22A shows sequential images of deploying a hybrid stent 130 with the sensory ring 124. FIG. 22B shows screenshots of the sensor signal plots before and after the deployment. FIG. 22C shows sequential images of deploying a silicone stent 130 with the sensory ring 124. Scale bars, 5 mm.

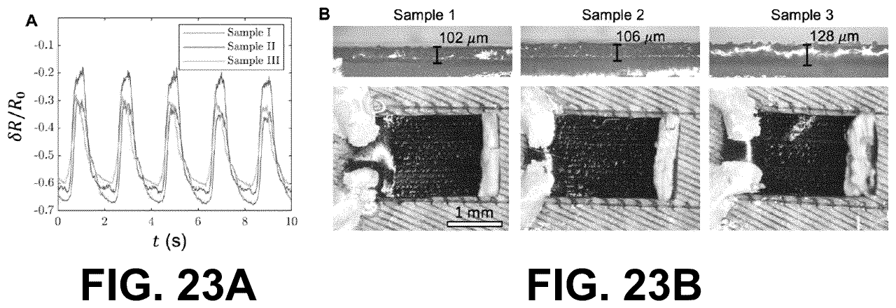

Then, at FIGS. 23A and 23B, shown is an example characterization of the variations of the mucus viscosity sensors 106. FIG. 23A shows δR/R₀ of three separately prepared viscosity sensors 106 as a function of time when actuated in syrup. Liquid viscosity: 9.6 Pa·s. Magnetic field 103 frequency: 0.5 Hz. Magnetic field 103 magnitude: 20 mT. FIG. 23B shows optical images of three separately prepared viscosity sensors 106 in the side and top views.

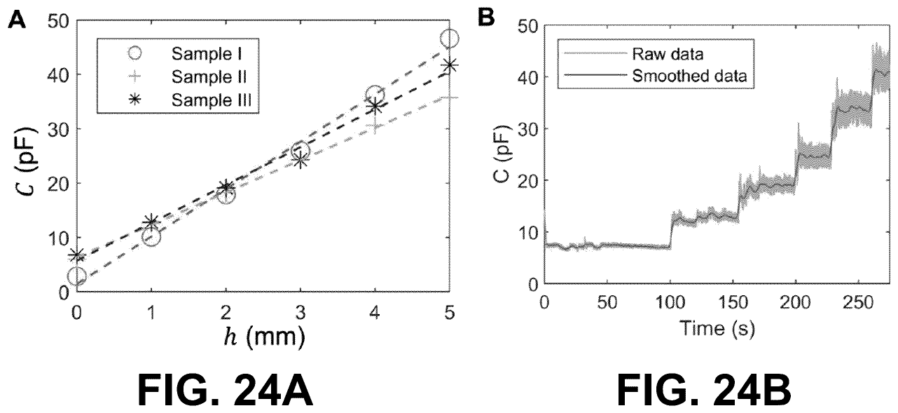

At FIGS. 24A and 24B, shown is an example characterization of the variations of the layer thickness sensors 109. FIG. 24A shows the capacitance of three separately prepared layer thickness sensors 109 as a function of the mucus layer thickness sensor 109. Mucus to water ratio is 1 to 8. FIG. 24B shows capacitance of a sensor 109 as a function of time when adding mucus to increase the layer thickness.

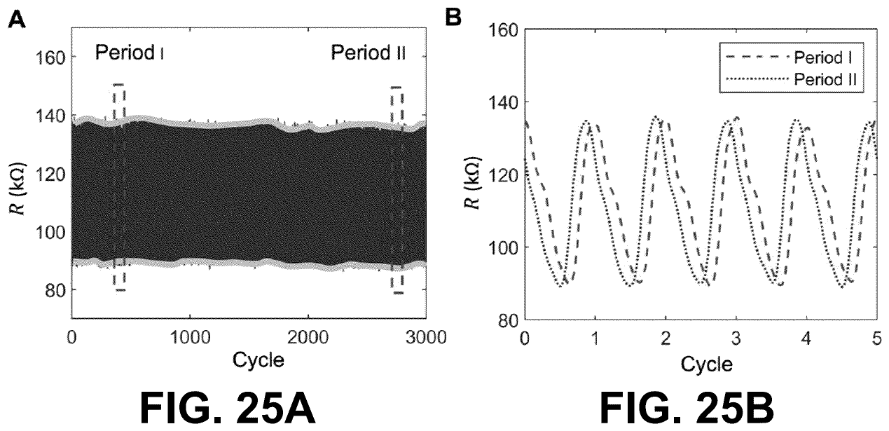

Moving to FIGS. 25A and 25B, shown is a characterization of the durability of a viscosity sensor 106. FIG. 25A shows sensor resistance as a function of the beating cycle when actuated by magnetic fields 103 in syrup. FIG. 25B shows zoomed-in plots of the sensor resistance as a function of the beating cycle. Magnetic field 103 frequency: 1 Hz. Magnetic field 103 magnitude: 20 mT. Liquid viscosity: 9.2 Pa·s.

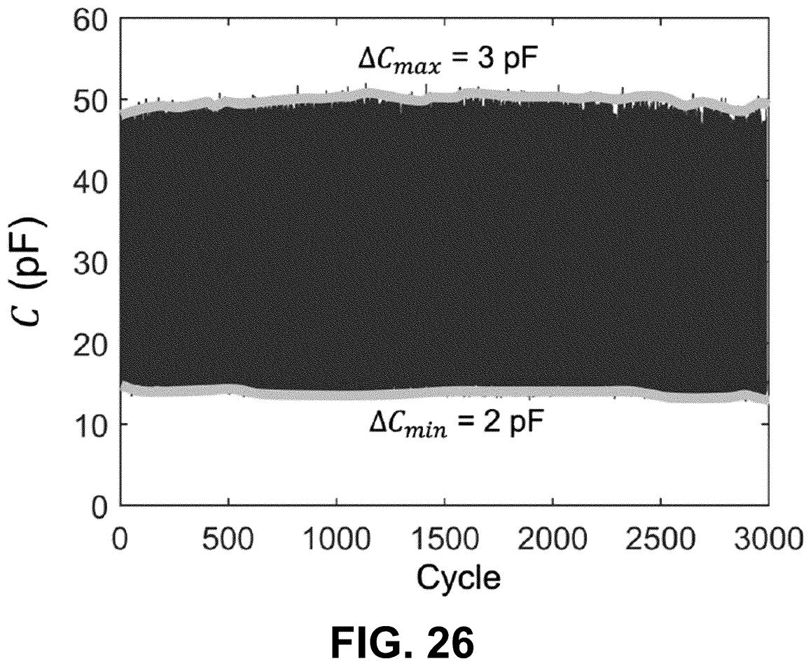

Next, at FIG. 26, shown is a characterization of the durability of a layer thickness sensor 109. Sensor capacitance is shown as a function of the bending cycle when actuated by magnetic fields 103 in mucus.

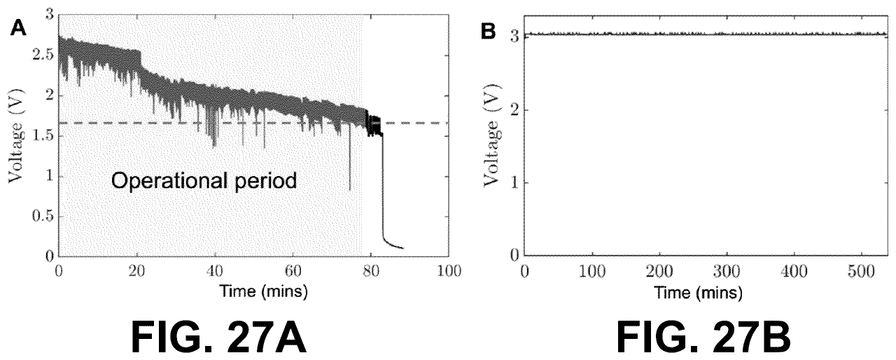

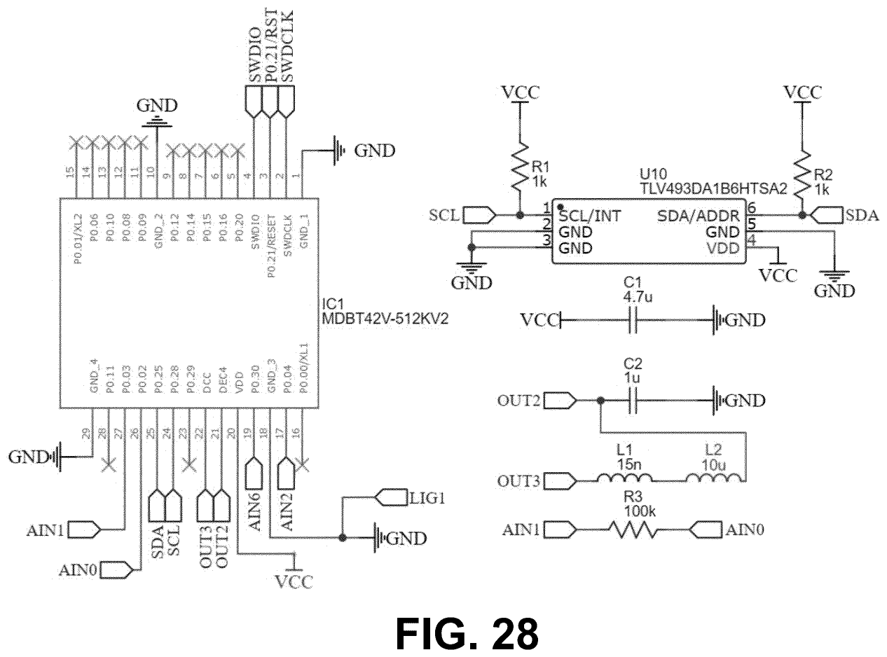

FIGS. 27A and 27B show a battery voltage as a function of time when continuously operating in different modes. FIG. 27A shows the voltage of two batteries 115 (LR626, 1.5 Volts, 18 mAh) when the BLE SoC 112 is in a working mode. Magnetic sensor: low power mode. Loop update rate: 5 Hz. FIG. 27B shows battery voltage when the BLE Soc 112 is in a sleeping mode. As the power consumption in the sleeping mode is negligible compared to the working mode, the device's lifespan is estimated using the operational period in FIG. 27A divided by ratio of the working mode time (10 seconds) over a full operation cycle duration (30 minutes).

In FIG. 28, shown is an example of circuit schematics of the fully integrated sensor. The mucus viscosity sensor 106 is connected to LIG1 and AIN1 (Analog Input 1). The mucus layer thickness sensor 109 is connected to AIN1 (Analog Input 2) and AIN6 (Analog Input 6).

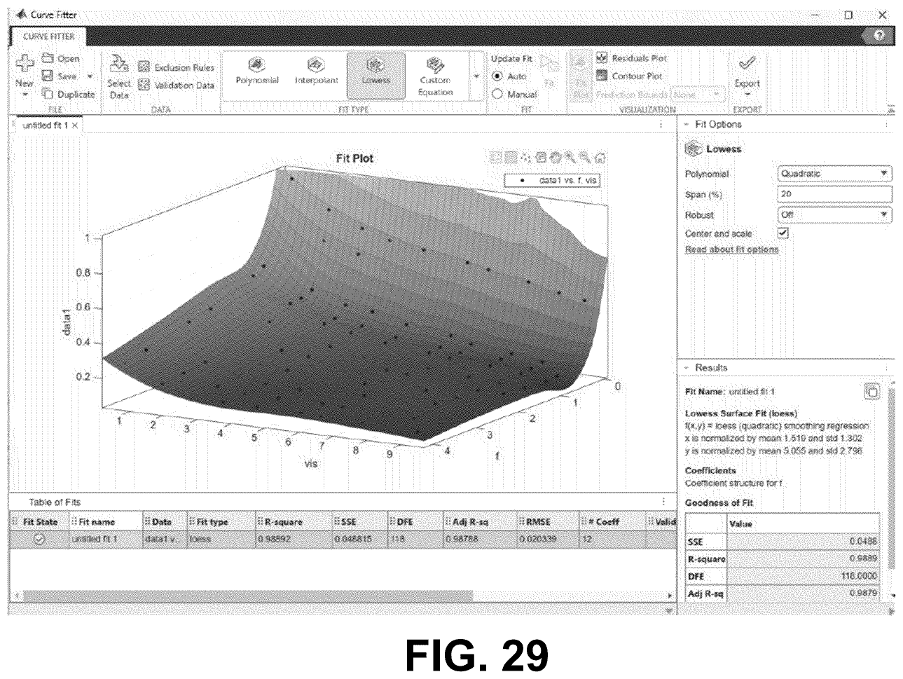

Turning to FIG. 29, shown is a parameter of the surface fitting function for the viscosity sensor 106.

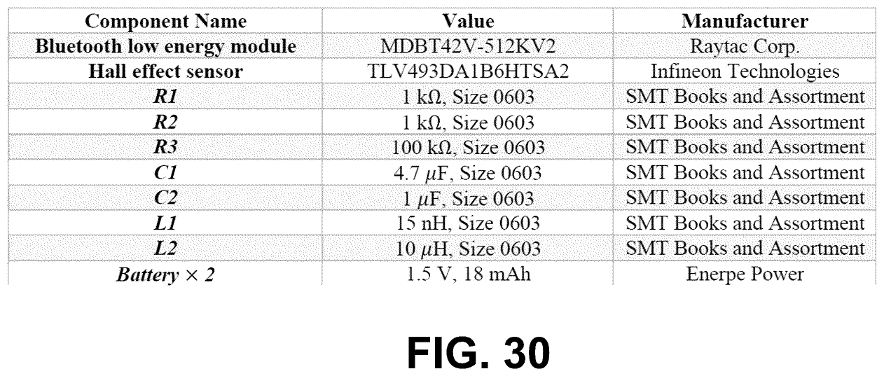

FIG. 30 shows a list of the components of the sensory ring 124.

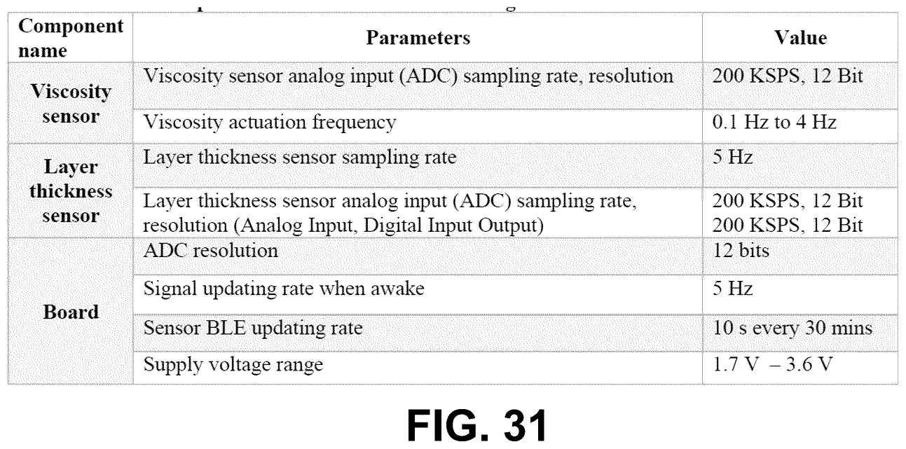

FIG. 31 shows technical parameters of the sensor with integration of the circuit.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

### Example A

### 1. Introduction

Airway stents (1, 2) play an important role for addressing diseases like central airway obstruction (3), as seen in conditions such as lung cancer, wherein the airway narrows from extrinsic compression. They are hollow, cylindrical tubes to provide crucial radial support (4), alleviating the effects of airway obstruction (5). However, a drawback of current airway stents is their interference with airway cilia, microscopic structures responsible for mucus transport (6), leading to mucus build-up. The accumulation of thick mucus in the airways poses challenges for patients by obstructing breathing and inducing inflammation, resulting in unpredictable episodes of airway distress and urgent bronchoscopy in cases of excessive mucus accumulation (7).

The properties of mucus such as viscosity and volume serve as important indicators of airway health and stent patency (8). For example, mucus viscosity reflects inflammation triggered by bacteria and other antigens (9) and the resulting viscous mucus could impede mucociliary transport and elicit airway obstruction. Meanwhile, the amount of mucus in the airway stent is important for determining further interventions as excessive mucus can cause clogging of the airway. Previous monitoring methods have relied on scheduled medical imaging checks such as Computational Tomography (10, 11) which has radiation, can be expensive, and must be performed in hospitals and therefore cannot provide continuous sensing. In addition, bronchoscopy (12, 13) is used for follow-up stent patency check but requires anesthesia and cannot provide continuous monitoring, either.

On one hand, monitoring mucus conditions by integrating sensors on implantable devices is a potential solution to allow continuous monitoring of the airway conditions and stent patency. Conventional stents, typically composed of inert materials such as silicone or metal, lack integrated sensors to monitor physiological conditions within the airway (14), constraining their effectiveness for both disease management and device monitoring. Existing efforts of integrating sensors on implantable devices inside the lumen of the human body have mostly focused on vascular (15) and esophageal stents (16). These sensor technologies have yet to be implemented in airway devices.

On the other hand, the lack of sensing mechanisms for wireless monitoring mucus conditions also prevents sensing mucus properties in the airway for patients with airway stents. First, previous efforts have predominantly focused on in vitro sensing (17), which are unsuitable for continuous long-term monitoring. Second, existing wireless devices used on the skin (18-22) provide long-term monitoring functions of the airway by sensing the vibration during breathing, but they cannot easily monitor the physiological conditions inside the airway. Lastly, existing implantable sensors (23) have demonstrated detecting temperature, flow, and pressure (24, 25), but the capability to assess mucus viscosity or volume is still missing. Previous research has explored integrating sensors into airway stents for assessing air flow and stent patency (26, 27), but these devices have issues of being bulky for long-term use due to their tethered nature. Therefore, it remains a grand challenge to develop miniature sensors for sensing mucus viscosity and volume continuously inside the airway.

Inspired by the sensing ability of biological cilia, the present disclosure provides for wireless sensing mechanisms in sensory artificial cilia for detecting mucus conditions, including viscosity and layer thickness. First, a mechanism is presented for artificial cilia to sense the viscosity of mucus (non-Newtonian) through fluid-structure interaction, implementing this mechanism on magnetically actuated artificial cilia with integrated flexible sensors. Second, a sensing mechanism for artificial cilia to sense the layer thickness of biofluids with self-calibration is presented, as well as reconfigurable sensitivity and range. The sensitivity and range of this sensor are adjusted by varying the angle of the external magnetic field. Third, to enable prolonged and wireless data access, Bluetooth Low Energy communication and onboard power are integrated, and create a wearable magnetic actuation system for actuating the sensors. Lastly, the sensor can be deployed independently or in conjunction with an airway stent, as shown within a trachea phantom and ex vivo sheep trachea. The ability to sense porcine mucus conditions is demonstrated using these sensory artificial cilia when integrated with versatile airway stents, which are widely used in various lung diseases leading to central airway obstruction. This real-time assessment of lung physiological conditions provides healthcare professionals with invaluable data, empowering them to make well-informed decisions regarding patient care.

### 2. Results

### 2.1. Working Principle of Sensory Artificial Cilia in an Airway Stent

Artificial cilia have been reported with promising fluid manipulation functions (28-30) at millimeter and micrometer scales. While integrating artificial cilia in airway stents has shown mucus transportation through fluid-structure interaction (27, 28), sensing mucus properties remains limited. As shown in FIG. 1A, the present disclosure can provide a cilia-inspired sensory device for sensing airway mucus conditions, which includes onboard sensors, data processing and transmission units, and a power module. As shown in FIG. 1B, a device according to various embodiments of the present disclosure can be integrated with an airway stent to continuously monitor advanced mucus conditions such as viscosity and layer thickness inside the human central airway, which are important biomarkers for disease diagnosis and stent patency monitoring. In addition to the onboard components, an external magnetic field, generated by a lightweight wearable system, plays a key role in enabling advanced sensing functions (for example, see section 2.4 below).

FIG. 1C illustrates the mechanism for sensing mucus viscosity of an artificial cilium. Inspired by biological cilia, the artificial cilium made of a magnetic composite dynamically interacts with the surrounding liquid when a rotating magnetic field is applied. The motion of the sensor is more obvious in a less viscous fluid when actuated by the same rotating magnetic field. Consequently, as shown in FIG. 1C, fluid viscosity can be interpreted from the deformation of the artificial cilium, estimated using the electrical resistance of a flexible strain-gauge produced by patterning laser-induced graphene (LIG). In addition, FIG. 1D shows the mucus layer thickness sensor and the mechanism for sensing mucus layer thickness by measuring the mucus-level dependent capacitance of a patterned capacitor. With the two conductive traces patterned parallel to each other with a narrow gap in between, the sensor's capacitance depends on the length of the gap filled by mucus. The mucus layer thickness is subsequently calibrated and measured by the sensor's capacitance using onboard circuits. The flexible hinge of the capacitor allows the sensor to be tilted remotely by magnetic fields such that the sensitivity and sensing range of the layer thickness sensor can be reconfigured on-demand by applying an external magnetic field.

The device is supported by wireless communication and onboard power. To enable wireless communication, FIG. 1E illustrates the sensor patch, which includes the Bluetooth LE System-on-a-Chip, the magnetic sensor, and connection ports to the power source and sensors. The sensor patch is prepared by laser-patterning a flexible circuit on Pyralux, fully encapsulated in PDMS for electrical insulation and biocompatibility. FIG. 1F shows the patch with integrated chips, peripheral circuits, and a battery, featuring the viscosity sensor, layer thickness sensor, magnetic sensor, and temperature sensor. The patch is attached to a TPU flexible ring as a backing layer to be implanted in the trachea as shown in FIG. 1G. In addition, FIG. 1H shows the system data flow chart, including collecting the analog input signals of the sensor's electrical resistance and capacitance using an onboard Bluetooth Low Energy System-on-a-Chip (nRF52832, Nordic Inc.), and sending the measured data to a cell phone or cloud via Bluetooth for further model-based prediction. Meanwhile, the external magnetic field at the sensor location is measured by an onboard Hall-effect sensor (TLV493D-A1B6, Infineon Technologies, Inc.) via the I2C communication protocol. Finally, as shown in FIG. 1I, the sensory ring with sensory artificial cilia can be further integrated with an airway stent, such as a self-expandable metal stent with a covered mesh, to monitor airway mucus conditions and stent patency. The mucus viscosity and layer thickness inside the central airway of a patient will be visualized on mobile devices or the cloud for disease diagnosis and patency monitoring.

### 2.2. Mechanism of Sensing the Viscosity of Non-Newtonian Fluids

FIG. 2 shows the fundamental mechanism of sensing the viscosity of non-Newtonian fluids. The sensory artificial cilium shown in FIG. 2A is fabricated using a laser patterning method (FIG. 7) and coated with polymer to allow a more robust sensing function compared with no encapsulation (see FIGS. 8A-8C and FIGS. 9A-9B). As shown in FIG. 2B, the magnetized viscosity sensor responds to an external magnetic field generated by a rotating magnet in an experimental setup (FIG. 10). The artificial cilium first bends by following the external magnetic field direction as the power stroke. As the bending curvature 1/ρ of the artificial cilium reaches to a critical state, a rotational snap-through behavior (31) causes the viscosity sensor to buckle back to its original state, as the recovery stroke. The sudden change of the resistance indicates the snap-through behavior when magnetic torque cannot withhold the elastic stress such that the magnetic cilium buckles back. During both strokes, fluid drag acts on the viscosity sensor, leading to different deformations in fluids of different viscosities. As the fluid drag depends on fluid viscosity, viscosity sensing can be achieved by monitoring the deformation of the viscosity sensor. As shown in FIG. 2C, the patterned conductive LIG is utilized to work as a strain sensing layer, allowing sensing strain by measuring the electrical resistance, which is inversely proportional to the bending curvature (see FIGS. 11A-11B, FIGS. 12A-12B).

The free body diagram of a sensory cilium is shown in FIGS. 11A-11B. According to the Euler-Bernoulli beam theory for large deflections (1), the moment-balance equation for a segment at [s, s+ds] at a quasi-static state is given by,

$$- EI \frac{{\partial }^{2} θ ( s , t )}{{\partial }^{2} s} = {A}_{cross} {τ}_{m} ( s , t ) - {F}_{h} ( s , t ) sin θ ( s , t ) + {F}_{v} ( s , t ) cos θ ( s , t ) , \qquad ( 1 )$$

where τₘ, E, I, and A_{cross} denote the magnetic torque per unit volume, the Young's Modulus, the second moment of area, and cross section area, respectively. Fₕ and Fᵥ are the internal forces applied on the horizontal and vertical directions. τₘ(s,t)=[R(θ(s,t))m(s)]×B(t), where R(θ(s,t)) is the rotation matrix. In addition, the force balance equation is given by

$${dF}_{h} = {f}_{dh} {A}_{cross} ds + {f}_{mh} {A}_{cross} ds , \qquad ( 2 )$$

$${dF}_{v} = {f}_{dv} {A}_{cross} ds + {f}_{mv} {A}_{cross} ds , \qquad ( 3 )$$

where f_{d} is the fluidic drag per volume. fₘₕ and fₘᵥ are the magnetic gradient pulling forces per volume in the horizontal and vertical directions. The external forces include magnetic gradient pulling forces and the fluid drag. The magnetic force per length is given by,

$${f}_{m} = \{ [ R ( θ ( s , t ) ) m ( s ) ] \cdot ∇ \} B ( t ) . \qquad ( 4 )$$

The fluid drag per length is given by,

$${f}_{h} = {f}_{d} sin θ ( s , t ) , \qquad ( 5 )$$

$${f}_{v} = {f}_{d} cos θ ( s , t ) , \qquad ( 6 )$$

where L is the length of the sensory cilium and f is the fluid drag per unit volume on an infinitesimal element. Because the artificial cilium forms a circular shape, the local drag coefficient C_{d} is obtained based on local curvatures and the relative velocity between the element and fluid flow, shown as,

$${f}_{d} = - \frac{1}{2} \frac{{\overline{C}}_{d} {ρ}_{f}}{{t}_{b}} \vert \frac{d {u}_{s} ( s , t )}{dt} \vert \frac{d {u}_{s}}{dt} , \qquad ( 7 )$$

where C̅_̅{̅d̅}̅=βC_{d}=β[−1.5 ln(Re)+7](2), with β as a coefficient related to the beam geometry, and the local Reynolds number Re is given by

$$R e = \frac{\vert {u}_{s} ( s , t ) \vert {ρ}_{f} d s}{{μ}_{f}} \cdot {ρ}_{f} , {t}_{b} and {μ}_{f}$$

denote the fluid density, artificial cilium thickness and the fluid dynamic viscosity, respectively. Finally, the model could be used to analyze the sensor behaviors in each liquid.

With a rotating magnetic field B(t), the deflection angle θ(s,t) of the sensory cilium depends on both the magnetic field and the fluid viscosity. The curvature of the sensory cilium

$$\frac{d θ ( s , t )}{d s}$$

is proportional to the length of the LIG sensor Lₛₑₙₛₒᵣ and therein the electrical resistance Rₛₑₙₛₒᵣ

$${R}_{sensor} = 2 {σ}_{s} {L}_{sensor} / {A}_{LIG} , where \qquad ( 8 )$$

$${L}_{s e n s o r} = [ L - {\int }_{0}^{L} \frac{t}{2} ( \frac{d θ}{ds} ) ds ] , {σ}_{s}$$

is the electric resistivity, and A_{LIG} is the cross-section of the LIG sensor trace.

We can use the sensor resistance as an indicator to the sensor behavior. With the same actuation frequency, a viscous fluid will result in a higher local drag coefficient C_{d} and thus a higher fluid drag. The net torque applied on the sensor will decrease as the magnetic actuation torque is always higher than the fluid drag. It will then lead to a decrease in

$$\frac{{\partial }^{2} θ ( s , t )}{{\partial }^{2} s} ,$$

which is the sensor curvature change over length. Thus, a viscous fluid will constrain the sensor deformation and thus set limit to its resistance range. Thus, a viscous fluid will constrain the sensor deformation and thus set limit to its resistance range. Similarly, a higher actuation frequency will lead to a higher fluid drag and a smaller change in the sensor resistance.

The theoretical model, initially developed to understand the sensor mechanism and guide the design of the viscosity sensor, builds on previous work (41). To validate the model's guidance, numerical simulations of the viscosity sensor in liquids were conducted with varying viscosities, actuated under magnetic fields of different frequencies as shown in FIGS. 12A-12B. The simulations demonstrated trends consistent with experimental results, offering valuable insights into sensor deformation under diverse conditions. However, it should be noted that the fluid-structure interaction between the viscosity sensor and mucus is highly complex due to the non-Newtonian behavior of the mucus.

The sensor deformation depends on the fluid viscosity, as well as the magnitude B and frequency f of the external rotating magnetic field. For example, a larger B field allows for a greater change in sensor resistance (FIG. 13). The magnetic field magnitude is fixed as 20 mT. This value is relatively easy to achieve at a typical stomal distance of 3.5 cm defined as the distance from the center plane of the human trachea to the skin. Moreover, FIGS. 2D-E investigate the sensing ability of the viscosity sensor in liquids of different viscosities when actuated by magnetic fields of different frequencies. For ease of comparison, the magnitude of the rotating magnetic field is fixed at 20 mT in all experiments. At an actuation frequency of 2 Hz, FIG. 2D plots the relative electrical resistance of the viscosity sensor as the output signal, defined as difference of the instantaneous electrical resistance R and its initial value R₀ when no external magnetic field is applied, normalized by R₀. The normalization of the resistance allows the elimination of effect of the electrical conductance coefficient. The peak-to-peak value SR of the sensor outputs is larger at a lower viscosity than at a higher viscosity. This occurs because the fluid drag is larger at a higher viscosity during the rapid snap-back motion, preventing the sensor from fully recovering to its initial state, as validated by extracting the shape of the viscosity sensor (FIG. 2E). This phenomenon remains consistent when actuating the viscosity sensor at different frequencies. FIG. 2F shows the sensor output at a frequency of 0.2 Hz, while FIG. 2G demonstrates that the envelope of the viscosity sensor becomes narrower in a liquid with a higher viscosity, further showcasing the effectiveness of the sensing mechanism.

The sensitivity and range of the viscosity sensor are fully characterized by performing tests in liquids of different viscosities and at different actuation frequencies. Herein, ‘sensitivity’ is defined as the slope of the sensor signal in response to changes in liquid viscosity. Additionally, the ‘measurement range’ refers to the range of liquid viscosities that can be estimated by the sensor based on interpolation from the training dataset. A mapping is established from the actuation frequency and sensor output signals to the liquid viscosity, which can be used to predict viscosities at different shear rates. Based on these training data, a predictive regression model can be constructed which maps from

$$pp ( \frac{δ R}{{R}_{0}} ) - the peak - to - peak value$$

δR normalized by R₀, and f to the fluid dynamic viscosity p as shown in FIG. 2H. It is assumed the material Youngs modulus does not degrade much across the implantation period. The calibration of the viscosity sensor needs to be completed once before the sensor is placed inside the trachea. Upon sensing, given f and

$$pp ( \frac{δ R}{{R}_{0}} )$$

within the range of the training data, the viscosity of an unknown fluid can be predicted using the trained model. Compared with a classification-based method (32), the regression model is computationally more efficient and easier to be used for predicting the shear-rate dependent viscosity of non-Newtonian fluids. The model landscape shows a larger functional gradient in the region with a relatively low viscosity and actuation frequency, as shown in FIG. 2H. Finally, in FIG. 2I, the viscosity sensor is characterized in liquids with viscosities ranging from 0.2 Pa·s to 7.3 Pa·s and at actuation frequencies from 0.1 Hz to 4 Hz. The extracted

$$pp ( \frac{δ R}{{R}_{0}} )$$

decreases monotonically as liquid viscosity increases. As in a high-frequency range, the curves in FIG. 2I are steeper indicating a higher sensitivity at higher magnetic actuation frequencies especially in the low-viscosity range. However, the sensing range is narrower when using a higher magnetic actuation frequency. Finally, in FIG. 2J, the sensing mechanism is validated by comparing the predicted viscosities at 0.3 Hz, 1 Hz, and 3.5 Hz with the viscosity values measured by a high-precision rheometer. The root mean square error was found to be 0.57 Pa·s at 3.5 Hz, which is lower than the 1.1 Pa·s and 1.2 Pa·s errors at 0.3 Hz and 1 Hz, respectively. The sensing accuracy is related to the sparsity of the training data. While the sensor demonstrates higher sensitivity at lower frequencies, it encounters challenges at higher frequencies, particularly when measuring liquids with high viscosity, which results in weaker sensor signals and a narrower sensing range.

To minimize the uncertainty of sensing mucus viscosity using the viscosity sensor in real-life applications, the effects of the magnitude and rotating plane of the magnetic field were investigated, as shown in FIGS. 3A-C. First, a calibration of a given viscosity sensor was performed using the process detailed in FIG. 2 to obtain a calibration model, as shown in FIG. 3A. The impact of the magnetic field magnitude on the sensing accuracy was then examined. As illustrated in FIG. 3B, when there is a 10% offset (2 mT and −2 mT) compared with the magnetic field magnitude (20 mT) used during the calibration, the average absolute sensing errors are approximately 0.13 Pa·s for liquids with the viscosities in the range from 2.5 Pa·s to 7.3 Pa·s. The average absolute sensing error slightly increases to 1.2 Pa·s when the liquid viscosity approaches 7.3 Pa·s. Therefore, it is desired that we can actively control the magnetic field magnitude to ensure precise estimation. In addition, in FIG. 3C, the rotating plane of the magnetic field was varied to introduce a small angle α between the rotating plane of the magnetic field and the y-z plane. The relative sensing error increases with fluid viscosity, but the relative errors remain relatively small (<8.4%), indicating the sensor's robustness to a small angle deviation in the rotating magnetic field due to the large width-to-length ratio (˜1), which helps avoid twisting.

Furthermore, using the calibrated viscosity sensor, its ability to sense the viscosity of mucus, a type of non-Newtonian fluid, was investigated. In FIG. 3D, it is demonstrated that the viscosity sensor can capture the shear-thinning behavior of porcine mucus. The sensor signals and actuation frequencies are used to predict mucus viscosity at different shear rates using the calibrated model. The mucus viscosity is predicted at different frequencies and compared with ground truth measurements from a rheometer. Despite prediction errors at higher shear rates, the predicted viscosity values exhibit a consistent trend with the ground truth data, effectively capturing the shear-thinning property of porcine mucus. Lastly, it was demonstrated that the continuous real-time sensing of time-varying mucus viscosity. As shown in FIG. 3E, the sensor is first placed in the air, then covered by pouring porcine mucus on top, which is subsequently diluted by adding water. The peak-to-peak electrical resistance of the sensor signal decreases when heating is applied, causing mucus dehydration. The video frames of this process shown in FIG. 3F further illustrates the sensing process and verifies the sensing ability. If the mucus does not fully cover the viscosity sensor, the sensor's readings may differ from those obtained when it is entirely covered. To mitigate this issue, the mucus thickness was assessed using a layer thickness sensor, which will guide the decision on whether to initiate viscosity measurement. Additionally, the viscosity threshold can be increased by either applying a stronger external magnetic field or reducing the magnetic field frequency. The proposed sensors are designed to sense mucus properties across a range close to the healthy condition and with inflammation (33).

The proposed viscosity sensor represents a significant advancement over existing technologies. Unlike previous work (32), a new magnetization profile has been developed for the sensor, enabling a substantial reduction in the external magnetic field required to actuate the viscosity sensor. Additionally, the focus has shifted to sensing non-Newtonian fluids, particularly mucus, rather than Newtonian fluids, utilizing flexible circuits. Finally, flexible sensors were designed and circuits that incorporate a novel prediction method using regression to accurately estimate mucus viscosity.

2.3. Mechanism for Sensing Mucus Layer Thickness with Reconfigurable Sensitivity and Range

To enable the sensing of mucus layer thickness and monitor the patency of an airway stent, a capacitor-based mucus thickness or layer thickness sensor capable of self-calibration was developed, as shown in FIG. 4A. The sensor is fabricated by attaching a capacitive sensing layer to a magnetic back layer (FIG. 14), allowing it to bend at different tilting angles when a magnetic field is applied enabling the ability to perform on-demand self-calibration. The sensor's capacitance changes as mucus, which has a much higher dielectric constant than air, fills the gap between the two conductive plates (FIG. 4B). FIG. 4C demonstrates the linear dependency of the sensor capacitance on the mucus thickness (see FIGS. 15A-15B, FIGS. 16A-16B), which enables the measurement of mucus thickness.

FIG. 14 illustrates the fabrication process of the flexible mucus layer thickness sensor. Initially, a UV laser patterns a thin layer of cured silver paste, forming two parallel electrical traces resembling a capacitor. Next, a thin layer of PDMS is spin-coated onto the electrodes to insulate them from liquids. Following this, a flexible hinge made from copper film connects the rigid capacitor to the circuitry. This design enables the sensor to respond to varying magnetic fields at different tilting angles. The fabricated layer thickness sensor has electrical traces of approximately 0.3 mm wide, with a gap of about 0.1 mm between them. The capacitor includes a flexible hinge that allows it to tilt in response to an external magnetic field. To prevent shorting, polymer encapsulation covers the electrodes and the copper tapes used for electrode-to-PCB connections. Additionally, for enhanced sensor sensitivity reconfiguration at a relatively large tilting angle, a small permanent magnet can be affixed to the sensor's tip.

The mucus layer thickness sensor is composed of two parallel plates bonded on a substrate (magnetic composite: NdFeB and Ecoflex 00-30) and encapsulated by PDMS or Ecoflex 00-30, as shown in FIGS. 15A-15B. When liquid fills in the gap between two electrodes, the capacitance of the whole device changes like existing layer thickness sensors (43) but with a tunable range and sensitivity. The height of the plate, the length of the plate, and the gap between the two plates are denoted by h₁, L_{c}, and d, respectively. A thin PDMS layer provides electrical insulation against the liquid environment, of which the thickness is t₁ on the side of the plates and t₂ on top of the substrate. ε₀, ε₁, ε₂ are defined as the dielectric constant of air, PDMS, and mucus, respectively.

The capacitance of the layer thickness sensor can be modeled using Finite Element Method. Based on charger conservation, the following is provided

$$∇ \cdot ( {ϵ}_{0} {ϵ}_{r} E ) = {ρ}_{v} , \qquad ( 9 )$$

$$- ∇ V = E , \qquad ( 10 )$$

$$D = {∈}_{0} {∈}_{r} E , \qquad ( 11 )$$

where ∈₀ and ∈ᵣ are the permittivity and relative permittivity, ρᵥ is the charge density.

The boundary conditions are given by,

$$n \cdot D = 0 , \qquad ( 12 )$$

$$V = 3.3 V , at the terminal , \qquad ( 13 )$$

$${V}_{0} = 0 , at the ground \qquad ( 14 )$$

where n is the surface normal, V and V₀ are the voltages of the terminal and the ground, respectively. The capacitance of the sensor when having a mucus wetted at a given height is given by C=Q/V. To provide insight into the thickness-dependent capacitance of the sensor, the capacitance of the layer-thickness sensor was calculated by varying the mucus layer thickness in Finite Element Analysis (FEA) as shown in FIGS. 16A-16B. The simulation results demonstrate a linear dependence of capacitance on layer thickness, which aligns with the experimental data. Intuitively, the layer thickness sensor is approximated as three capacitors connected in parallel (44) including 1) the part above the red dashed line with mucus covered, denoted as C_{Bottom}, and 2) part with no mucus covered but filled with air, denoted as C_{Top}. The total capacitance is given by,

$${C}_{sensor} = {C}_{Top} ( {L}_{c} - {l}_{m} ) + {C}_{Bottom} ( {l}_{m} ) . \qquad ( 15 )$$

lₘ=h/sin α_{c} with h as the layer thickness of the mucus and α_{c} as the tilting angle of the sensor relative to the sensor ring surface. As C_{Top}∝(Lₑ−lₘ), then Cₛₑₙₛₒᵣ is given by,

$${C}_{sensor} = k {l}_{m} + {C}_{0} = \frac{k}{sin {α}_{c}} h + {C}_{0} . \qquad ( 16 )$$

Therefore, the capacitance of the mucus layer thickness sensor Cₛₑₙₛₒᵣ is linearly dependent on the mucus layer thickness.

In addition, the sensor is coated with PDMS for biocompatibility and preventing the sensor from shorting (FIGS. 17A-17B). When there is no encapsulation layer, the mucus will short the capacitor sensor (FIG. 18), making an encapsulation layer necessary. However, even with an encapsulation layer, mucus of different mucin concentrations will affect the capacitance-based layer thickness readings. To investigate and address this issue, the layer thickness sensor was tested on mucus with different mucin concentrations using mixtures with various water-mucin weight ratios, as shown in FIG. 4D. Within a sensing range of 5 mm, the slopes between capacitance and mucus thickness are distinct due to the different dielectric constants of the testing fluids.

To address this issue, a self-calibration process leveraging the external magnetic field is proposed to allow the calibration of the layer thickness sensor in unknown mucus. This is particularly important when implanting the sensor inside the human airway where the mucus concentration and dielectric constant are typically unknown. As shown in FIGS. 4E and 4F, the calibration process includes the following steps. First, the sensor is initially not covered by mucus at all, resulting in a minimum capacitance value Cₘᵢₙ (˜5 pF). Then, it is submerged in the mucus by tilting the magnetic field, obtaining the maximum capacitance value Cₘₐₓ (˜60 pF), which represents the upper limit when mucus thickness is about 5 mm. Based on the linear relationship between the sensor capacitance and mucus thickness, the sensor coefficients are obtained. Subsequently, the sensor is tilted up by external magnetic fields to point perpendicular to the mucus layer. The excessive mucus on the sensor flows back gradually over time. Lastly, the mucus layer thickness is measured using the calibrated sensor model, as shown in FIG. 4G.

An additional important advantage of controlling the sensor's tilting angle is that the sensitivity and sensing range can be dynamically adjusted by tuning the tilting angle with an external magnetic field. In FIGS. 4H and 4I, mucus of a given thickness results in different sensor capacitance outputs at various tilting angles, as the capacitance-thickness slope changes with the tilting angle. This allows for dynamic reconfiguration of the sensing ability in different scenarios for more precise measurement of mucus properties. For a relatively thin layer of mucus, the tilting angle can be controlled to be smaller for more accurate measurement, while increasing the tilting angle to 90° in a relatively thick layer of mucus allows for a larger measurement range. It is important to note that the surface properties of the coating influence wetting behavior due to both viscosity and capillary effects (34). The Ecoflex 00-30 coating, with its larger water contact angle, facilitates the backflow of mucus when the sensor is tilted at various angles. Moving forward, the surface properties of the sensor should be further optimized to enhance sensitivity to mucus layer thickness and to ensure precise reflection of this thickness.

2.4. Demonstration of Integrating with Airway Stents and Sensing Mucus Properties

A wearable magnetic actuation system was developed for portable actuation of the sensory artificial cilia, as illustrated in FIG. 5A. The system includes a rotating magnet controlled by a servo motor for translation and a DC motor for rotation. These motors are managed by an embedded controller (Arduino Nano 33 BLE Sense), enabling wireless control via mobile devices. FIG. 5B shows a prototype of the wearable magnetic actuation system mounted on a human chest phantom. To characterize the magnetic field generation capability, the magnetic field generated by the wearable system was measured at different locations. The wearable system can generate a magnetic field up to 40 mT at 2.5 cm and operate at a frequency up to 5 Hz. FIG. 5C demonstrates that the magnetic field magnitude varies with the y and z positions of the magnet when placed symmetrically about the y-z plane. Additionally, the magnetic field waveform is characterized at a specific location. At a point directly beneath the central magnet at d_{z}=3.5 cm, the rotating magnetic field exhibits similar magnitudes for both the y and z components, as shown in FIG. 5D. With this magnetic actuation system, FIG. 5E depicts the motion of the viscosity sensor and the layer thickness sensor when actuated by the wearable system at d_{z}=3.5 cm. Furthermore, the magnetic field magnitude and frequency can be controlled on demand by adjusting the rotating speed of the magnet and its y position (FIG. 5F). The wearable magnetic system weighs approximately 686 grams, including batteries. Bluetooth Low Energy-based wireless communication is integrated into the system, allowing for remote control of the spinning speed and magnetic field magnitude (FIGS. 19A-19D). In addition, it was shown that the on-board magnetic field sensor can be used to provide feedback information of the external magnetic field (FIGS. 20A-20F) and allow adjustment of the external magnetic actuation unit to ensure the actuation magnetic field is as desired.

As the sensory ring is designed for implantation inside the human trachea, a customized delivery tool was developed for stent deployment by modifying a flexible delivery tool used for self-expandable hybrid stents. This delivery tool consists of a handle, a flexible tube, and a customized head to hold the sensory ring and stent, as shown in FIG. 6A. During the delivery process, the sensory ring is first compressed into the head of the tool, as illustrated in FIG. 6B. The tool is then inserted into a trachea phantom. Once the sensory ring is pushed out of the head of the delivery tool, it expands due to the stored elastic energy, as shown in FIG. 6C. The measurement process for mucus viscosity and layer thickness is further outlined in FIG. 6D. Initially, both the viscosity and mucus layer thickness sensors are in a horizontal position when no magnetic field is applied (FIG. 6D (i)). As mucus is added to the stent, it gradually submerges the sensors. An online self-calibration procedure for the layer thickness sensor is performed (FIG. 6D (ii)). When the capacitance of the layer thickness sensor reaches its maximum value, a constant magnetic field is applied to lift the thickness sensor to a specific angle (60 degrees) out of the mucus. The thickness measurement is obtained as the mucus layer thickness stabilizes on the thickness sensor, as shown in FIG. 6D (iii). After completing the mucus layer thickness measurement, a rotational magnetic field is applied to actuate the viscosity sensor within the mucus (FIG. 6D (iv)). Throughout the experiments, the sensor signals are transmitted to a mobile device or PC via Bluetooth.

The capability of integrating the sensory artificial cilia with airway stents for sensing mucus viscosity and layer thickness was developed using a trachea phantom. FIGS. 6E and 6F show sensory rings integrated with a hybrid airway stent and a silicone airway stent, respectively. The hybrid airway stent with a sensory ring can be delivered using a customized flexible delivery tool by folding the hybrid stent and compressing the sensory ring. Meanwhile, the silicone airway stent with a sensory ring can be delivered into the trachea using a rigid bronchoscope.

To demonstrate the sensing ability in a trachea phantom, the data of the sensed time-varying mucus viscosity and layer thickness was plotted using the sensor ring inside an airway phantom in FIGS. 6G and 6H, respectively. The two sensors will operate sequentially. First, the mucus layer thickness will be measured using the layer thickness sensor, which includes online calibration capabilities. If the measured thickness is sufficient to fully submerge the viscosity sensor, the viscosity sensor will then be activated by a rotating magnetic field. FIG. 6G illustrates the resistance change of the viscosity sensor during the deployment and viscosity measurement. Initially, the resistance fluctuates slightly due to vibration when the sensor is folded, but it quickly stabilizes as the sensory ring is deployed and expands. When mucus is added and a rotational magnetic field is applied, the sensor effectively responds to changes of viscosity over time promising for monitoring the viscosity changes due to inflammation or dehydration.

Moreover, FIG. 6H shows the capacitance change during mucus layer thickness measurement. The mucus accumulation is detected by an abrupt change in capacitance, which quickly rises to its maximum value while the sensor is lying down for the self-calibration process. After the sensor is lifted, the mucus gradually slips away from the sensor, allowing for the computation of mucus layer thickness based on the current mucus coverage and tilting angle. When the mucus layer thickness reaches a threshold, an alarm will be triggered so that further intervention could be carried out in time. To accurately determine the sensor's bending angle within the body, the onboard magnetic sensor will measure the magnetic field and calculate the angle based on a pre-established mapping. This mapping correlates the magnetic sensor readings with the sensor's angle, which can be calibrated as demonstrated in FIGS. 21A-21B. To measure mucus thickness when the sensor is inside the body, the magnetically controlled bending angle of the layer thickness sensor will be leveraged. In the experiments, the process will begin by measuring the maximum capacitance (Cₘₐₓ) with the sensor positioned flat, fully submerged in the mucus. Then, a magnetic field will be applied to tilt the sensor, with the tilting angle determined by the detected magnetic field angle. The sensor's tilting angle will be gradually increased until the capacitance reaches Cₘₐₓ. This approach avoids sensor saturation while ensuring the reading is sufficiently large. If the sensor still registers maximum capacitance even at a 90-degree angle, it indicates that the mucus layer thickness exceeds the sensor's length.

Additionally, it was demonstrated that the combination of the sensory ring with different types of airway stents and its resilience in a sheep trachea. FIG. 6I shows the delivery of a hybrid stent and a sensory ring into a sheep trachea ex vivo, demonstrating the resilience of the sensory ring during the delivery process. For clear visualization and illustration, the deployment processes of the sensor ring with a hybrid stent and a silicone stent in a transparent trachea phantom (FIGS. 22A-22C). The deployment process and result of a sensory ring with a hybrid stent, a sensory ring alone, and a sensory ring with a silicone stent inside a sheep trachea are further recorded and verified using an X-ray medical imaging machine as shown in FIGS. 6J-L. The results further demonstrate the feasibility of visualizing the sensory airway stent inside the animal organs.

### 3. Discussion

In summary, the present disclosure has culminated in the development of a novel airway stent equipped with integrated artificial cilia. These cilia possess the remarkable ability to sense various mucus conditions, including viscosity, thickness, and temperature, thereby holding promise for monitoring stent patency. The viscosity sensor operates on the principle of an artificial cilium actuated by an external magnetic field, while a flexible strain gauge sensor measures the curvature of the cilium. For mucus thickness sensing, a capacitor was employed that can be tilted by an external magnetic field for self-calibration and reconfigurable sensing range and sensitivity. Furthermore, temperature data is also captured using an onboard sensor. The artificial cilium sensors are activated by a customized wearable magnetic actuation system, facilitating real-time monitoring of lung physiology and mucus properties. By continuously gathering data on mucus viscosity, quantity, and other pertinent parameters, these sensors provide continuous monitoring of airway conditions and stent patency for timely interventions.

The variance among the sensors has been investigated. As shown in FIGS. 23A-23B, the thickness of the viscosity sensor is the primary factor influencing the sensor's bending angle when actuated in the same fluids under identical magnetic fields. Despite these variations, all viscosity sensors can be calibrated after fabrication by actuating them in liquids with known viscosities. The layer thickness sensors do not require pre-calibration, even though their capacitance may vary with changes in mucus thickness, as demonstrated in FIGS. 24A-24B. These sensors are designed for online calibration using magnetic field actuation. After implantation, they can be calibrated and subsequently used to measure mucus thickness post-calibration.

Repeatability and durability are also crucial for both sensors. To assess these properties, cyclic actuation experiments were conducted. First, a viscosity sensor was actuated in a liquid for N=3000 cycles while monitoring its electrical resistance. As shown in FIG. 25A, the peak-to-peak value of the electrical resistance signal remains relatively constant, with zoomed-in plots in FIG. 25B further illustrating this stability. A layer thickness sensor was actuated in mucus, controlling its bending angle between zero and 90 degrees for N=3000 cycles while simultaneously measuring the sensor's capacitance. As shown in FIG. 26, the minimum and maximum capacitances remain relatively stable, demonstrating the sensor's repeatability. Additionally, the online calibration mechanism allows for compensation of any sensor degradation through recalibration.

Additionally, optimizing the electronic device's battery life can be achieved by fine-tuning the sensor update frequency. For example, adjusting the update interval to every 30 minutes for a 10-second measurement could extend the device's operational lifespan to 10 days (see FIGS. 27A-27B). To further extend the lifetime, wireless charging units (16, 19, 24) can be integrated for remote powering of the stent. Moreover, improving biocompatibility can be achieved by encapsulating the electronic board with polyimide tapes on both surfaces and coating the magnetic sensor with PDMS. To further enhance biocompatibility, the viscosity sensor should be coated, with magnetic particles potentially coated in SiO₂ (35). Additionally, the device's biocompatibility could be further improved by applying a parylene-C coating. Verification of biocompatibility can be achieved through in vitro testing using cell viability analysis. Lastly, the anchoring force of the sensor ring in an animal trachea needs to be quantified when bonding with a hybrid stent. To further quantify stent migration and other complications when implanted in an animal model, the in vivo performance of artificial cilia can be assessed using Computational Tomography.

Further, multimodal sensors (36) capable of detecting temperature, pressure, hydration, stent migration, and air flow can be integrated to enable comprehensive monitoring of mucus properties and airway conditions. This continuous monitoring could allow for the early detection of changes in mucus properties or the onset of complications (37, 38), facilitating timely interventions (39) and personalized close-loop therapy (40). By closely tracking mucus properties and airway health, a device according to the present disclosure can support tailored treatment strategies for each patient's unique needs, optimizing therapeutic outcomes and preventing disease exacerbations.

### 4. Materials and Methods

Fabrication of sensory artificial cilia for viscosity sensing. The fabrication process for the sensory artificial cilia commences with the preparation of laser-induced graphene (LIG). Initially, a layer of polyethylene terephthalate (PET) was affixed to a glass slide, followed by the spin-coating of Polyvinyl alcohol (PVA) onto the PET layer to establish an adhesive surface. After curing the PVA at 100° C. for 5 minutes, a layer of polyimide (PI) tape was applied onto the PVA layer. Subsequently, a CO2 laser was employed to induce graphene formation from the PI tape, utilizing parameters set to 20% power, 50% speed, and 1000 PPI. Once the graphene was induced from the PI, a magnetic composite comprising NdFeB particles (MQFP 15-7, diameter: 5 μm) and Polydimethylsiloxane (PDMS) at a weight ratio of 2:1 was spin-coated onto the graphene layer at 2700 RPM for 1 minute. The coated material was then cured on a hot plate at 100° C. for 10 minutes. The resulting LIG-polymer layer, along with the PI layer, is carefully removed from the glass slide. A heating process using LPKF U4 equipment was applied to facilitate the separation of the LIG-polymer from the PI layer. Subsequently, the transferred LIG was patterned into the desired sensory artificial cilium shape. This artificial cilium was affixed to a backing layer using Ecoflex 00-30. Connection between the LIG on the artificial cilium and conductive traces was established using carbon paste. A thin layer of Ecoflex 00-30 was then applied to the LIG to serve as an encapsulation layer. Additionally, the traces were encapsulated using Ecoflex 00-30 for added protection. Finally, the sensory artificial cilium was magnetized, allowing the integration into the sensor ring.

Fabrication of sensory artificial cilia for mucus layer thickness sensing. Initially, a layer of magnetic composite substrate composed of Ecoflex 00-30 and NdFeB (with a weight ratio of 1:2) was prepared. Following this, a Polyimide (PI) film was applied to enhance sensor rigidity and electrode adhesion. The electrodes, fabricated from silver paste, were cured on a hot plate at 150° C. for 20 minutes. Subsequently, electrode patterning for capacitors was executed using a UV laser machine (LPKF U4, from LPKF Laser & Electronics North America). To prevent shorting of the capacitor to surrounding fluids, a layer of Polydimethylsiloxane (PDMS) was spin-coated onto the electrodes at 4000 RPM for 1 minute, then cured at 100° C. for 10 minutes. Ecoflex 00-30 might be used as an alternative coating material for more viscous liquid as the adhesion between the coating and mucus is smaller compared with PDMS. Next, Laser-Induced Graphene (LIG) patches were affixed to the electrodes and electrical traces using silver paste, serving as flexible conductive hinges. The sensor assembly was then magnetized at 2.2 Tesla. For insulation, the LIG patches and silver paste were encapsulated within Ecoflex 00-30. This encapsulation process ensures electrical integrity and environmental resilience for the sensor system.

Fabrication of the electronic circuit for computation and communication. Custom circuits were designed to accommodate an MDBT42V-P512KV2 chip from Raytac Corp (FIG. 30). and a 3-axis magnetometer (TLV493D-A1B6) from Infineon Technologies, AG. These circuit boards were designed using Electronic Design Automation (EDA) software, specifically EasyEDA, as illustrated in FIG. 28. Subsequently, the PCBs were fabricated with precision using the LPKF U4 system from LPKF Laser & Electronics North America. During the PCB assembly phase, a paste mask was employed to accurately apply solder paste onto the copper pads, ensuring proper connection for the nRF52832 microprocessor. Following this, other essential components such as capacitors were meticulously soldered onto the custom circuits. For a comprehensive listing of these components, refer to FIG. 30. Programming of the Bluetooth Low Energy (BLE) System-on-Chip (SoC) was accomplished using an nRF52 Development Kit from Nordic Semiconductor. The program was initially compiled in Arduino IDE 1.8.19, utilizing the Adafruit Bluefruit library, before being uploaded to the BLE SoC via the nRF52 Development Kit. To provide power supply, a battery board equipped with two alkaline batteries (LR626, 1.5 Volts, 18 mAh) was affixed to the custom boards, ensuring seamless functionality. The sampling rate and resolution data of the sensory board are listed in FIG. 31.

Preparation of the customized delivery tool. The customized flexible delivery tool was prepared by adapting a commercial stent delivery tool designed for self-expandable stents. A conical outer shell, boasting a maximum inner diameter of 16 mm and a length of 25 mm, was 3D printed using Thermoplastic Polyurethane (TPU) and securely affixed to the tip of the stent delivery tool. During the deployment process, the sensory ring was securely bonded to a metal stent and adeptly folded to conform to the dimensions of the cone-shaped structure. Simultaneously, the metal stent was meticulously folded within the existing plastic shell of the delivery tool, ensuring a compact and streamlined configuration. Upon reaching the targeted location within the trachea, the sensor ring was released, popping up together with the metal stent. This seamless integration ensured precise positioning and optimal functionality of the sensory ring within the trachea, facilitating accurate monitoring and data collection.

Experimental setup for testing the viscosity sensor. The viscosity sensor was interfaced with copper wires to enable resistance measurement. Subsequently, it was positioned within a transparent container, suspended approximately 3 cm above two rotating permanent magnets. These magnets are measured with dimensions of 25 mm by 25 mm by 25 mm (N45, from SuperMagnetMan). Once the sensor was submerged in the liquid of interest, viscosity measurements were conducted using LabVIEW 2020, National Instruments. This setup allowed for precise and reliable assessment of viscosity layer thickness, facilitating comprehensive analysis of the fluid's properties.

Wearable magnetic actuation system. The system comprises housing, rails, a ground plate, slider crank mechanism, translation table, shaft stabilizer, motor mount, and sleeves for the magnetic module. The system housing was fabricated using 3D printing technology and polylactic acid (PLA) filament. Additionally, the top plate and driver component board were precision-cut from 3-mm thick plywood using a laser. For the magnetic actuation unit, two NdFeB magnets with radial magnetization (diameter: 25 mm, length: 25 mm, N45 grade, from Applied Magnets) were snugly housed within PLA sleeves, designed to prevent magnet rotation. These sleeves were carefully aligned and joined together using a vice, ensuring correct orientation, and then fused together through plastic welding. The rotation of the permanent magnets was achieved using an encoder gear motor (N20) controlled by a DC motor driver (L298N). Meanwhile, a servo motor (MG996R) was employed to regulate the position of the permanent magnets through a sliding-crank linkage mechanism. To manage the operation of both the DC motor and servo motor, a wirelessly controlled embedded controller (Arduino Nano 33 BLE Sense) was utilized. Power was supplied by two rechargeable batteries (3.7 V, 5000 mAh, Model: 18650, from Tokeyla) for the motors, with an additional 9V battery dedicated to powering the embedded controller. The magnetic field at the sensor location depending on the position of the sensor and the magnet inside the wearable magnetic actuation system.

Preparing viscous liquids. Different viscous liquids were prepared by mixing syrup (Karo Light Corn Syrup, dynamic viscosity μ=7.8 Pa·s) with different amount of water. For example, the syrup-water mixture of a weight ratio of 100:1, has the viscosity of μ=2.8 Pa·s. Their viscosities were measured by a viscometer (NDJ-8S, Bonvoisin). The mucus was prepared by mixing porcine mucin (Chem-impex International Inc.) with water according to different weight ratios. The mixtures were then stirred for 1 hour at room temperature (23° C.).

Fitting function of the viscosity sensor. The fitting function used during the calibration in Matlab was ‘Loess-Quadratic’, also known as locally weighted polynomial regression. The parameters used in the fitting functions are shown in FIG. 29, with a span of 20% of the dataset and quadratic polynomial implemented. All data were equally weighted.

Data collection on the Bluetooth LE SoC and in Matlab. Arduino code specifically tailored for the Adafruit Bluefruit nRF52 Board was employed to program the nRF52832 chip. Additionally, a dedicated Arduino library for the 3D magnetic sensor (TLV493D-A1B6) from Infineon Technologies Inc. was utilized to activate the on-board magnetic sensor, ensuring accurate and reliable data collection. To streamline data acquisition and processing, the Bluetooth Toolbox in Matlab 2022a (Mathworks Inc.) was used, which provided the functions for Bluetooth Low Energy communication, enabling seamless integration with the Adafruit Bluefruit nRF52 Board.

### REFERENCES

1. E. Folch, C. Keyes, Airway stents. Ann Cardiothorac Surg 7, 27383-27283 (2018).
2. N. Guibert, H. Saka, H. Dutau, Airway stenting: Technological advancements and its role in interventional pulmonology. Respirology 25, 953-962 (2020).
3. A. Ernst, D. Feller-Kopman, H. D. Becker, A. C. Mehta, Central Airway Obstruction. Am J Respir Crit Care Med 169, 1278-1297 (2004).
4. G. N. Herlitz, D. I. Sternberg, R. Palazzo, S. Arcasoy, J. R. Sonett, Treatment of Bronchomalacia in Cystic Fibrosis by Silicone Stent. Annals of Thoracic Surgery 82, 2268-2270 (2006).
5. N. Guibert, H. Saka, H. Dutau, Airway stenting: Technological advancements and its role in interventional pulmonology. Respirology 25, 953-962 (2020).
6. X. M. Bustamante-Marin, L. E. Ostrowski, Cilia and Mucociliary Clearance. Cold Spring Harb Perspect Biol 9, a028241 (2017).
7. J. V. Fahy, B. F. Dickey, Airway Mucus Function and Dysfunction. New England Journal of Medicine 363, 2233-2247 (2010).
8. C. M. Evans, J. S. Koo, Airway mucus: The good, the bad, the sticky. Pharmacol Ther 121, 332-348 (2009).
9. S. K. Lai, Y. Y. Wang, D. Wirtz, J. Hanes, Micro- and macrorheology of mucus. Adv Drug Deliv Rev 61, 86-100 (2009).
10. B. Xiao, Y. Xu, S. Edwards, L. Balakumar, X. Dong, Sensing Mucus Physiological Property In Situ by Wireless Millimeter-Scale Soft Robots. Adv Funct Mater 34, 2307751 (2024).
11. P. A. de Jong, N. L. Müller, P. D. Paré, H. O. Coxson, Computed tomographic imaging of the airways: relationship to structure and function. European Respiratory Journal 26, 140-152 (2005).
12. H. J. Lee, et al., Airway stent complications: the role of follow-up bronchoscopy as a surveillance method. J Thorac Dis 9, 4651-4659 (2017).
13. A. Crutu, P. Baldeyrou, “Stent Monitoring and Care” in Normal and Pathological Bronchial Semiology, (Elsevier, 2019), pp. 163-174.
14. I. Costanzo, D. Sen, L. Rhein, U. Guler, Respiratory Monitoring: Current State of the Art and Future Roads. IEEE Rev Biomed Eng 15, 103-121 (2022).
15. J. Vishnu, G. Manivasagam, Perspectives on smart stents with sensors: From conventional permanent to novel bioabsorbable smart stent technologies. Med Devices Sens 3, e10116 (2020).
16. C. Zhang, et al., Wirelessly powered deformable electronic stent for noninvasive electrical stimulation of lower esophageal sphincter. Sci Adv 9 (2023).
17. K. R. Atanasova, L. R. Reznikov, Strategies for measuring airway mucus and mucins. Respir Res 20, 261 (2019).
18. M. Liu, et al., Non-Invasive Flexible Electro-Mechanical Sensors for Human Respiratory Monitoring and Chronic Disease Management. Adv Mater Technol 9, 2302010 (2024).
19. J. Y. Yoo, et al., Wireless broadband acousto-mechanical sensing system for continuous physiological monitoring. Nature Medicine 2023 29:12 29, 3137-3148 (2023).
20. Z. Che, et al., Speaking without vocal folds using a machine-learning-assisted wearable sensing-actuation system. Nature Communications 2024 15:1 15, 1-11 (2024).
21. T. Jiang, et al., Wearable breath monitoring via a hot-film/calorimetric airflow sensing system. Biosens Bioelectron 163, 112288 (2020).
22. H. Jeong, et al., Closed-loop network of skin-interfaced wireless devices for quantifying vocal fatigue and providing user feedback. Proc Natl Acad Sci USA 120, e2219394120 (2023).
23. M. Veletić, et al., Implants with Sensing Capabilities. Chem Rev 122, 16329-16363 (2022).
24. K. Kwon, et al., A battery-less wireless implant for the continuous monitoring of vascular pressure, flow rate and temperature. Nature Biomedical Engineering 2023 7:10 7, 1215-1228 (2023).
25. M. Lin, H. Hu, S. Zhou, S. Xu, Soft wearable devices for deep-tissue sensing. Nature Reviews Materials 2022 7:11 7, 850-869 (2022).
26. L. J. L. Ruiz, J. Zhu, L. Fitzgerald, D. Quinn, J. Lach, Capacitive Sensing for Monitoring Stent Patency in the Central Airway in 2021 43rd Annual International Conference of the IEEE Engineering in Medicine & Biology Society (EMBC), (IEEE, 2021), pp. 5441-5445.
27. L. Ciaffoni, et al., In-airway molecular flow sensing: A new technology for continuous, noninvasive monitoring of oxygen consumption in critical care. Sci Adv 2 (2016).
28. T. ul Islam, et al., Microscopic artificial cilia—a review. Lab Chip 22, 1650-1679 (2022).
29. H. Gu, et al., Magnetic cilia carpets with programmable metachronal waves. Nat Commun 11 (2020).
30. W. Wang, et al., Cilia metasurfaces for electronically programmable microfluidic manipulation. Nature 605, 681-686 (2022).
31. X. Dong, et al., “Bioinspired cilia arrays with programmable nonreciprocal motion and metachronal coordination” (2020).
32. J. Han, et al., Actuation-enhanced multifunctional sensing and information recognition by magnetic artificial cilia arrays. Proc Natl Acad Sci USA 120, e2308301120 (2023).
33. P. Prasher, et al., Targeting mucus barrier in respiratory diseases by chemically modified advanced delivery systems. Chem Biol Interact 365, 110048 (2022).
34. L. Chen, E. Bonaccurso, Effects of surface wettability and liquid viscosity on the dynamic wetting of individual drops. Phys Rev E Stat Nonlin Soft Matter Phys 90, 022401 (2014).
35. Y. Kim, G. A. Parada, S. Liu, X. Zhao, Ferromagnetic soft continuum robots. Sci Robot 4, 7329 (2019).
36. X. Ni, et al., Automated, multiparametric monitoring of respiratory biomarkers and vital signs in clinical and home settings for COVID-19 patients. Proc Natl Acad Sci USA 118, e202661 0118 (2021).
37. K. Murase, S. Neri, R. Tachikawa, K. Tomii, Tracheal stent placement via a tracheostomy for tracheal stenosis after inhalation injury. Burns 36, e132-e135 (2010).
38. H. J. Lee, et al., Airway stent complications: the role of follow-up bronchoscopy as a surveillance method. J Thorac Dis 9, 4651-4659 (2017).
39. K. J. Bayfield, et al., Time to get serious about the detection and monitoring of early lung disease in cystic fibrosis. Thorax 76, 1255-1265 (2021).
40. Y. Wang, S. Sharma, F. Maldonado, X. Dong, Wirelessly Actuated Ciliary Airway Stent for Excessive Mucus Transportation. Adv Mater Technol 8, 2301003 (2023).
41. G. Z. Lum, et al., Shape-programmable magnetic soft matter. Proc Natl Acad Sci USA 113, E6007-E6015 (2016).
42. X. Dong, et al., “Bioinspired cilia arrays with programmable nonreciprocal motion and metachronal coordination” (2020).
43. B. Kumar, G. Rajita, N. Mandal, A review on capacitive-type sensor for measurement of height of liquid level. Measurement and Control (United Kingdom) 47, 219-224 (2014).
44. X. Huang, et al., High-stretchability and low-hysteresis strain sensors using origami-inspired 3D mesostructures. Sci Adv 9 (2023).

Therefore, the following is claimed:

1. A device, comprising:
a tubular member having a radially external side and a radially internal side;
a viscosity sensor disposed on the radially internal side of the tubular member and configured to measure a viscosity of a fluid within an airway of a subject in response to a bending motion of the viscosity sensor; and
a layer thickness sensor disposed on the radially internal side of the tubular member and configured to measure a thickness of the fluid within the airway of the subject in response to a tilting motion of the layer thickness sensor,
wherein the layer thickness sensor is configured to engage in the tilting motion, the viscosity sensor is configured to engage in the bending motion, or both in response to an application of an external magnetic field.

2. The device of claim 1, further comprising one or more of a temperature sensor or a magnetic sensor disposed on the radially internal side of the tubular member.

3. The device of claim 1, further comprising a wireless communication module disposed on the radially internal side of the tubular member and configured to wirelessly communicate with one or more computing devices.

4. The device of claim 1, further comprising an airway stent coupled to one or more ends of the tubular member.

5. The device of claim 1, wherein the external magnetic field has a frequency of approximately 0.1 Hz to approximately 5 Hz, a magnitude of approximately 5 mT to approximately 70 mT, or both.

6. The device of claim 1, wherein the viscosity sensor comprises a strain-gauge configured to measure a deformation of the viscosity sensor based at least in part on an electrical resistance of the strain-gauge, wherein the viscosity of the fluid is calculated based at least in part on the deformation.

7. The device of claim 1, wherein the layer thickness sensor comprises a capacitor configured to measure a capacitance of the capacitor, wherein the thickness of the fluid is calculated based at least in part on the capacitance.

8. The device of claim 1, further comprising at least one rechargeable battery disposed on the radially internal side of the tubular member, wherein the application of the external magnetic field charges the at least one rechargeable battery.

9. A method of monitoring an airway of a subject, comprising:
applying an external magnetic field to an artificial cilium implanted within the airway of the subject, wherein the artificial cilium comprises a layer thickness sensor configured to tilt in response to the external magnetic field;
measuring a capacitance using the layer thickness sensor; and
calculating a thickness of a fluid in the airway of the subject based at least in part on the capacitance.

10. The method of claim 9, wherein the external magnetic field has a magnitude of approximately 5 mT to approximately 70 mT.

11. The method of claim 9, wherein the artificial cilium further comprises a viscosity sensor, the method further comprising:
determining the thickness of the fluid is greater than a threshold, wherein the threshold is based at least in part on the viscosity sensor;
applying a second external magnetic field, wherein the viscosity sensor is configured to bend in response to the second external magnetic field;
measuring a deformation of the viscosity sensor; and
calculating a viscosity of the fluid in the airway of the subject based at least in part on the deformation.

12. The method of claim 9, wherein measuring the capacitance using the layer thickness sensor further comprises:
measuring a maximum capacitance without the application of the external magnetic field;
applying the external magnetic field to tilt the layer thickness sensor to a tilting angle, wherein the tilting angle is based at least in part on an angle of the external magnetic field; and
gradually increasing the tilting angle until the capacitance reaches the maximum capacitance.

13. The method of claim 11, further comprising diagnosing a condition of the airway of the subject based at least in part on the thickness of the fluid, the viscosity of the fluid, or both.

14. The method of claim 11, wherein the second external magnetic field has a frequency of approximately 0.1 Hz to approximately 5 Hz and a magnitude of approximately 5 mT to approximately 70 mT.

15. The method of claim 9, further comprising alerting a user when the thickness of the fluid is equal to or greater than a threshold.

16. A system, comprising:
an implant, comprising:
a tubular member having a radially external side and a radially internal side;
a viscosity sensor configured to measure a viscosity of a fluid in an airway of a subject in response to a bending motion of the viscosity sensor; and
a layer thickness sensor configured to measure a thickness of the fluid within the airway of the subject in response to a tilting motion of the layer thickness sensor,
wherein the layer thickness sensor is configured to engage in the tilting motion, the viscosity sensor is configured to engage in the bending motion, or both in response to an application of an external magnetic field;
a magnetic actuator, comprising:
a magnet coupled to a slider crank mechanism; and
at least one motor configured to actuate the slider crank mechanism and the magnet;
a computing device in wireless communication with the implant and the magnetic actuation device, comprising a processor and a memory; and
machine-readable instructions stored in the memory that, when executed by the processor, cause the computing device to at least:
activate the magnetic actuation device to tilt the layer thickness sensor based at least in part on a first external magnetic field;
calculate, using the layer thickness sensor, the thickness of the fluid within the airway of the subject based at least in part on the application of the first external magnetic field;
activate the magnetic actuation device to bend the viscosity sensor based at least in part on a second external magnetic field; and
calculate, using the viscosity sensor, the viscosity of the fluid within the airway of the subject based at least in part on the application of the second external magnetic field.

17. The system of claim 16, wherein the first external magnetic field has a magnitude of approximately 5 mT to approximately 70 mT, and the second external magnetic field has a frequency of approximately 0.1 Hz to approximately 5 Hz and a magnitude of approximately 5 mT to approximately 70 mT.

18. The system of claim 16, wherein the viscosity sensor comprises a strain-gauge configured to measure a deformation of the viscosity sensor based at least in part on an electrical resistance of the strain-gauge, wherein the viscosity of the fluid is calculated based at least in part on the deformation.

19. The system of claim 16, wherein the layer thickness sensor comprises a capacitor configured to measure a capacitance of the capacitor, wherein the thickness of the fluid is calculated based at least in part on the capacitance.

20. The system of claim 16, further comprising at least one rechargeable battery disposed on the radially internal side of the tubular member, wherein the application of the external magnetic field charges the at least one rechargeable battery.
